(19) United States
(12) Patent Application Publication
(10) Pub. No.: US 2015/0276757 A1
(43) Pub. Date: Oct. 1, 2015

(54) WHEAT PROTEOMIC MICROARRAY FOR BIOMARKER DISCOVERY

(71) Applicant: THE TRUSTEES OF COLUMBIA UNIVERSITY IN THE CITY OF NEW YORK, New York, NY (US)

(72) Inventor: Armin Alaedini, New York, NY (US)

(73) Assignee: THE TRUSTEES OF COLUMBIA UNIVERSITY IN THE CITY OF NEW YORK, New York, NY (US)

(21) Appl. No.: 14/414,221

(22) PCT Filed: Aug. 23, 2013
(86) PCT No.: PCT/US2013/056354

§ 371 Date: Jan. 12, 2015

Related U.S. Application Data
(60) Provisional application No. 61/692,418, filed on Aug. 23, 2012.

(51) Int. Cl. G01N 33/68

(52) U.S. Cl. CPC G01N 33/6854; G01N 2333/415; G01N 2570/00

(57) ABSTRACT

This invention pertains to the preparation of arrays containing the proteome of wheat, including gluten and non-gluten proteins. Antibodies to wheat gluten have been shown to be elevated, not only in celiac disease and wheat allergy, but also in neuropsychiatric disorders, such as schizophrenia, bipolar disorder, and autism. The array would be able to detect specific patterns of antibody reactivity to gluten proteins that are unique to each disease and which may have utility as biomarkers. The array may also have use in detecting patterns of cross-reactive autoantibodies in other disorders.

### CROSS-REFERENCE TO RELATED APPLICATIONS

This application claims the benefit of priority of U.S. Ser. No. 61/692,418, filed Aug. 23, 2012. The entire content and disclosure of the preceding application are incorporated by reference into this application.

### FIELD OF THE INVENTION

This invention pertains to the preparation and uses of arrays containing the proteome of wheat, including gluten and non-gluten proteins.

### BACKGROUND OF THE INVENTION

Glutens are the major storage proteins of wheat and related cereals, comprising over 70 different molecules in any given wheat variety (Dupont et al., 2011). The main classes of gluten include α/β-gliadins, γ-gliadins, ω-gliadins, high molecular weight glutenins, and low molecular weight glutenins (Jabri et al., 2005). Gluten sensitivity can be defined as a state of heightened immunologic reaction to gluten proteins, which may be accompanied by increased levels of antibodies against them. Heightened immune reactivity to gluten is recognized and understood best in the context of celiac disease, an autoimmune disorder primarily targeting the small intestine, and wheat allergy (Ludvigsson et al., 2013). The humoral immune response in celiac disease also includes antibodies to deamidated sequences of gliadin and to the autoantigen transglutaminase 2 (TG2), which are highly specific and sensitive serologic markers of the condition (Briani et al., 2008). Celiac disease is also closely linked with genes that code for human leukocyte antigens (HLA) DQ2 and DQ8 (Qiao et al., 2012).

However, some individuals complain of symptoms in response to ingestion of “gluten”, without histologic or serologic evidence of celiac disease or wheat allergy (Sapone et al., 2012; Volta et al., 2011). The term “non-celiac gluten sensitivity” (NCGS) has been suggested for this condition. Currently, there is very limited information about NCGS, as the antigenic triggers for the condition remain completely unknown, the mechanism is unclear, and no biomarkers are available to identify affected individuals. The lack of increased antibody response to transglutaminase enzyme or association with HLA-DQ2/DQ8 in NCGS indicates that the immune response to gluten in NCGS is significantly different from celiac disease, probably having a different mechanism that is less dependent on presentation by HLA-DQ2/DQ8 molecules and the deamidating activity of TG2 enzyme (Volta et al., 2011). The antibody responses to gluten in NCGS patients may target a unique set of proteins and epitopes that can be utilized to understand the disease mechanism and identify novel biomarkers for the disease condition. Thus, there is a need to characterize the molecular specificity of the immune response to wheat proteins in NCGS.

Immune responses to wheat proteins are also observed in other diseases, for example, in schizophrenia or autism, and determining the molecular specificity of the immune response to wheat proteins in these other diseases may similarly provide novel biomarkers for these diseases. Schizophrenia is a chronic and highly debilitating mental illness affecting about 1% of the U.S. population (National Institute of Mental Health, 2011). A critical barrier to a better understanding of the condition, accurate diagnosis and follow up of patients, and discovery of more effective therapies has been the lack of specific disease biomarkers. Recently published reports point to increased circulating levels of antibody to gluten in nearly one third of individuals with SZ (Cascella et al., 2009; Dickerson et al., 2010; Jin et al., 2010).

Regarding autism, although the etiology and pathogenesis of autism are poorly understood, there is evidence that immune system abnormalities are associated with symptoms in a substantial number of affected individuals (Onore et al., 2012). In addition, several studies have evaluated gastrointestinal (GI) symptoms and defects in GI barrier function in patients with autism (Wang et al, 2011; Adams et al., 2011; D'Eufemia et al., 1996; de Magistris et al., 2010). A possible association between autism and celiac disease was first discussed over 40 years ago (Dohan, 1969; Goodwin et al., 1969). Although some studies have pointed to higher frequency of celiac disease, family history of celiac disease, or elevated antibody to gliadin among autistic children (Barcia et al., 2008; Valicenti-McDermott et al., 2008; Vojdani et al., 2004), others have not supported these findings (Pavone et al., 1997). Diets that exclude gluten are becoming increasingly popular in the autism community, but their effectiveness has not been proven in controlled and blinded studies (Elder, 2008). Despite years of speculation and immense interest by families of affected children regarding the potential connection between autism and gluten sensitivity, no well-controlled study has been performed to determine the levels of immune reactivity to gluten in patients, to characterize the antigenic specificity of this immune response, or to assess its pathogenic relevance to autism.

The invention described herein would provide a systematic approach to characterize the molecular specificity of the immune response to wheat proteins in various diseases, thereby generating data that can be utilized to understand the disease mechanism and identify novel biomarkers for the diseases.

### SUMMARY OF THE INVENTION

In one embodiment, this invention provides a method of determining the molecular specificity of antibody response to gluten and non-gluten proteins of wheat in a group of patients, the method comprises the steps of: (i) preparing a composition comprising gluten and non-gluten proteins from wheat; (ii) generating a gluten microarray using the composition obtained in (i); and (iii) generating profiles of antibody binding to target proteins or peptides on the array of (ii), wherein the antibodies are obtained from patients or control subjects, and the binding profile of antibodies from said patients as compared to those from control subjects will demonstrate the molecular specificity of antibody response to gluten and non-gluten proteins of wheat in said group of patients.

In one embodiment, the composition obtained in (i) above comprises recombinant gluten and non-gluten proteins from wheat. In another embodiment, the composition comprises extracts of gluten and non-gluten proteins from wheat, e.g. extracts derived from intact gluten proteins or gluten digest. In one embodiment, the extracts include intact proteins, proteins after enzymatic digestion, or peptides.

In one embodiment, the above extracts are prepared by fractionation of proteins or peptides by high resolution chromatographic separation methods. In another embodiment, the preparation of extracts comprises 2-D fractionation of proteins or peptides.

In one embodiment, the above method further comprises the step of identifying the target proteins or peptides by mass spectrometry-assisted peptide mass mapping. In another embodiment, the above method further comprises the step of identifying antigenic determinants on the target proteins by epitope mapping.

In one embodiment, the patients mentioned above display increased antibody response to wheat proteins and/or are having a disease such as celiac disease, wheat allergy, dermatitis herpetiformis, non-celiac gluten sensitivity, schizophrenia, bipolar disorder, autism, or sporadic ataxia.

### BRIEF DESCRIPTION OF THE DRAWINGS

FIG. 1 shows comparison of levels of IgG and IgA antibody to gliadin in children with autism, their unaffected siblings, and unrelated healthy controls. Boxed segments represent the middle 50% of the data. Whiskers indicate the range of data. Large horizontal bars indicate mean value of the data. **=p<0.01.

FIG. 2. shows comparison of levels of antibody to A) gliadin, B) deamidated gliadin fusion peptide, and C) human TG2 in autistic children, with and without GI symptoms. Boxed segments represent the middle 50% of the data. Whiskers indicate the range of data. Large horizontal bars indicate mean value of the data. **=p<0.01.

FIG. 3 shows the chromatographic separation of the various wheat extractions, as well as the digests.

FIG. 4 show print layout for the wheat proteomic microarray, demonstrating the position of the gluten proteins, non-gluten albumin/globulin proteins, and gluten digest peptides.

FIG. 5 shows mass spectrometry-assisted identification of gluten proteins separated by HPLC and printed on the microarray. A) HPLC chromatogram and numbering of eluted gluten peaks. B) Protein bands identified by mass spectrometry. Numbers correspond to the HPLC peak number.

FIG. 6 shows mass spectrometry-assisted identification of non-gluten proteins separated by HPLC and printed on the microarray. A) HPLC chromatogram and numbering of eluted gluten peaks. B) Protein bands identified by mass spectrometry. Numbers correspond to the HPLC peak number.

FIG. 7 shows wheat proteomic microarray prototype. A) Map of the printed gluten protein and digest fractions, as well as control spots. B) Scan of printed array at a wavelength showing all spotted fractions. FIGS. 7C and 7D show respectively IgA and IgG antibody reactivity of serum from celiac disease patient towards the gluten proteins and peptides on the array. FIGS. 7E and 7F show respectively IgA and IgG antibody reactivity of serum from a healthy subject towards the gluten proteins and peptides on the array.

FIG. 8 shows images of IgG and IgA binding from patients and controls (celiac disease, dermatitis herpetiformis, schizophrenia, and healthy control) to chromatographically separated proteins and peptides printed on the glass microarrays.

FIG. 9 shows pattern of antibody reactivity to chromatographic fractions of Butte 86 gluten proteins. (A) RP-HPLC chromatogram for Butte 86 gluten protein extract and the collected 96 fractions, (B-D) pattern of antibody reactivity to gluten protein chromatographic fractions for a representative CD patient (B), a representative patient with schizophrenia and elevated antibodies to gluten (C) and a representative healthy control (D) expressed as mean intensity of array spot signal (Fmean) after subtraction of the median of Fmean of all buffer spots for each array (array buffer median).

FIG. 10 shows pattern of antibody reactivity to 96 chromatographic fractions of digested gluten proteins. (A) RP-HPLC chromatogram for digested Cheyenne gluten proteins, (B-D) pattern of antibody reactivity to digested gluten protein chromatographic fractions for a representative CD patient (B), a representative NCGS patient with schizophrenia (C) and a representative healthy control (D). Antibody reactivity is expressed as mean intensity of array spot signal (Fmean) after subtraction of the median of Fmean of all array buffer spots for each array (array buffer median).

### DETAILED DESCRIPTION OF THE INVENTION

The present invention provides a systematic approach to assess the molecular specificity of the observed immune response to gluten in various diseases using a gluten microarray system. In general, gluten proteins from different wheat cultivars can be purified into different fractions, for example, through two-dimensional chromatography, and localized on functionalized glass slides in microarray format. Antibody reactivity to spotted fractions can be detected by an array scanner using, for example, fluorescent-tagged secondary antibodies. Target proteins of interest can be identified by various methods such as LC-MS/MS-assisted peptide mass mapping. Antigenic determinants of significant target proteins can further be determined by epitope mapping.

Basically, a protein array contains an array of immobilized protein spots. Each spot can contain a homogeneous or heterogeneous set of “bait” molecules. A spot on the array may display an antibody, a cell or phage lysate, a recombinant protein or peptide, a drug, or a nucleic acid. The array is queried with a probe (e.g. labeled antibody or ligand), or an unknown biologic sample (e.g., cell lysate or serum sample) containing analytes of interest. By tagging the query molecules with a signal-generating moiety, a pattern of positive and negative spots is generated. For each spot, the intensity of the signal is proportional to the quantity of applied query molecules bound to the bait molecules. An image of the spot pattern is captured, analyzed, and interpreted.

In general, protein microarrays are printed using the same technology used for DNA microarrays, but the protein array layout is vastly different from a typical DNA array. Both printing technologies transfer sample fluid from a microtiter plate onto a substratum, usually a coated glass slide. The substratum requirements for protein arrays are (1) high binding capacity, (2) minimum effect on the protein structure, and (3) low background. Nitrocellulose coated glass slides are a common substratum for protein arrays. Proteins bind to nitrocellulose via electrostatic interactions in an irreversible manner. Protein arrays may also be printed in sector formats. A sector array consists of multiple small pads of substratum on a slide. A reservoir placed around each sector permits a different antibody to be used for probing the samples. The sector format miniaturizes the array, providing an increased signal/noise ratio.

Wheat flour protein composition contains a complex mixture of similar but distinct proteins (Dupont et al., 2011). The major water-insoluble protein fraction, comprised largely of glutenin polymers and gliadin monomers, is often referred to as gluten; these proteins are also categorized among the proline- and glutamine-rich cereal storage proteins known as prolamins. High molecular weight glutenin subunits (HMW-GS) and low molecular weight glutenin subunits (LMW-GS) are linked by disulfide bonds between Cys residues to form polymers that contribute strength and elasticity to flour doughs, whereas the monomeric gliadins contribute to dough viscosity and extensibility. A single hexaploid wheat variety contains 6 genes for HMW-GS, 20 or more LMW-GS genes, 29 or more gamma-gliadins genes, up to 150 alpha-gliadin genes and at least 5 omega-gliadin genes, although not all of these genes are expressed. In addition, some proteins with gliadin-like sequences have an odd number of Cys residues and can be linked to the glutenin polymer. Flour also contains smaller amounts of other storage proteins such as globulins and triticins, proteins such as amylase and protease inhibitors that may protect against insects and fungi, and small amounts of various enzymes. Recently, a majority of abundant flour proteins from a single wheat cultivar have been identified and related to individual gene sequences (Dupont et al., 2011). These data and other related databases would help in distinguishing the target proteins identified by the method of the present invention. In other words, potential target proteins from wheat include, but are not limited to, one or more of the following: gluten proteins such as gliadins and glutenins, as well as non-gluten proteins such as amylase/trypsin inhibitors, serine protease inhibitors, purinins, globulins, and farinins etc.

One of ordinary skill in the art would readily generate a microarray and analyze the profiles of antibody binding according to the method described herein. Protein arrays comprise a wide variety of experimental designs. In one embodiment, antibodies may be arrayed as capture molecules to perform microspot ELISA-type experiments for quantitative profiling of protein expression or for detecting the presence of their antigens in complex lysates. Recombinant or purified proteins can be immobilized to study protein-protein interaction or to probe sera for the presence of specific antibodies. Alternatively, complex tissue or cell lysates (or fractions thereof) can be immobilized and probed with a number of antibodies to profile the presence of antigens in many samples under identical condition.

Various methods can be used to detect binding profiles on the microarray. A variety of protein array labels and amplification chemistries are available. These include fluorescent, radioactive, luminescent, and colorimetric readouts. Chromogenic, fluorometric, and luminescent detection methods may be used with an adequate signal/noise ratio. Amplification can be achieved by enzymatic cleavage of colorimetric, luminescent, and fluorescent substrates. In one embodiment, detecting antibody binding on the microarray using fluorescent dyes is very convenient as it is simple, has high spatial resolution as well as very high sensitivity. Commonly used fluorophores include, but are not limited to, Cy3, Cy5, corresponding Alexa- and DY-fluorophores, phycoerythrin and others. Infra-red fluorophores such as IR800 have also been used with excellent results. In general it has been observed that longer wavelength fluorophores such as Cy5 (and analogs) or IR800 are often advantageous. Many biomolecules present in blocking reagents and samples have an inherent autofluorescence and will bind to the surface thus contributing to background. This phenomenon is less pronounced when using red and far-red wavelengths for detection. For some applications signal amplification will be necessary. In one embodiment, systems employing horseradish peroxidase (HRP) are used. With either HRP or alkaline phosphatase (AP), both chemiluminescent or chromogenic substrates can be used.

One of ordinary skill in the art would readily recognize that the present invention can be easily adapted or coupled to electronic or computerized machine and software for the steps of generating a microarray, generating profiles of antibody binding, and/or analyzing the profiles of antibody binding. In one embodiment, an automated imaging system can be used for automated acquisition, compilation, and analysis of images of antibody binding profiles.

In one embodiment, the present invention provides a method of determining the molecular specificity of antibody response to gluten and non-gluten proteins of wheat in a group of patients, the method comprises the steps of: (i) preparing a composition comprising gluten and non-gluten proteins from wheat; (ii) generating a gluten microarray using the composition obtained in (i); and (iii) generating profiles of antibody binding to target proteins or peptides on the array of (ii), wherein the antibodies are obtained from patients or control subjects, and the binding profile of antibodies from said patients as compared to those from control subjects will demonstrate the molecular specificity of antibody response to gluten and non-gluten proteins of wheat in said group of patients.

In one embodiment, the composition obtained in (i) above comprises recombinant gluten and non-gluten proteins from wheat. In another embodiment, the composition comprises extracts of gluten and non-gluten proteins from wheat, e.g. extracts derived from intact gluten proteins or gluten digest. In one embodiment, the extracts include intact proteins, proteins after enzymatic digestion, or peptides. In another embodiment, the extracts are derived from digesting the gluten or non-gluten proteins with one or more of pepsin, trypsin, and chymotrypsin.

In one embodiment, the above extracts are prepared by fractionation of proteins or peptides by high resolution chromatographic separation methods. In another embodiment, the preparation of extracts comprises 2-D fractionation of proteins or peptides.

In one embodiment, the above method further comprises the step of identifying the target proteins or peptides by mass spectrometry-assisted peptide mass mapping. In another embodiment, the above method further comprises the step of identifying antigenic determinants on the target proteins by epitope mapping.

In one embodiment, the patients mentioned above display increased antibody response to wheat proteins. In another embodiment, the patients mentioned above are having a disease such as celiac disease, wheat allergy, dermatitis herpetiformis, non-celiac gluten sensitivity, schizophrenia, bipolar disorder, autism, or sporadic ataxia.

In one embodiment, the target proteins or peptides identified by the above method are gluten proteins. Examples of gluten proteins include, but are not limited to, gliadins and glutenins. In one embodiment, the identified target proteins or peptides are gliadins or related proteins. In another embodiment, the identified target proteins or peptides are glutenins or related proteins.

In one embodiment, the present invention also provides uses of the above identified target gluten proteins as biomarkers for various diseases or conditions.

In one embodiment, the target proteins or peptides identified by the above method are non-gluten proteins. Examples of non-gluten proteins include, but are not limited to, amylase inhibitors, trypsin inhibitors, serine protease inhibitors, purinins, globulins, and farinins. In one embodiment, the identified target proteins or peptides are amylase inhibitors or related proteins. In another embodiment, the identified target proteins or peptides are trypsin inhibitors or related proteins. In another embodiment, the identified target proteins or peptides are serine protease inhibitors or related proteins. In another embodiment, the identified target proteins or peptides are purinins or related proteins. In another embodiment, the identified target proteins or peptides are globulins or related proteins. In another embodiment, the identified target proteins or peptides are farinins or related proteins.

In one embodiment, the present invention also provides uses of the above identified target non-gluten proteins as biomarkers for various diseases or conditions.

### Example 1

### Molecular Specificity of Antibody Response in Non-Celiac Gluten Sensitivity

It is hypothesized that the antibody response to gluten in NCGS patients differs significantly from celiac disease, targeting a unique set of proteins and epitopes that can be utilized to understand the disease mechanism and identify novel biomarkers of the condition. The specific aims of this example represent a systematic approach to characterizing the molecular specificity of the immune response to wheat proteins in NCGS using an innovative microarray system, as follows.

Aim 1. To Construct a Wheat Proteomic Microarray Containing the Full Set of Immunogenic Gluten and Non-Gluten Proteins.

Gluten and non-gluten proteins from two different U.S. wheat cultivars will be separated and fractionated by reversed phase HPLC and localized on functionalized glass slides in microarray format.

Aim 2. To Characterize the Molecular Specificity of the Antibody Response in NCGS.

The constructed proteomic microarray system will be utilized to generate accurate data about the molecular specificity of the immune response to gluten/non-gluten proteins of wheat in NCGS. Target proteins of interest will be identified by LC-MS/MS-assisted peptide mass mapping.

### Rationale

The last decade has seen a dramatic rise in public awareness and interest in gluten, use of gluten-free products, and attribution of a broad range of symptoms by patients to “gluten sensitivity” (Sapone et al., 2012). A recent double-blinded, randomized, and placebo-controlled study of non-celiac/non-allergic individuals has shown that gluten intake is associated with increase in certain symptoms in some individuals, suggesting that true NCGS may in fact exist (Biesiekierski et al., 2011). Another recently published study showed that patients with antibodies to gliadin and celiac-specific HLA markers (DQ2 and/or DQ8) have more gastrointestinal symptoms than antibody-negative individuals (Ruuskanen et al., 2011). The gastrointestinal symptoms in these individuals were mild to severe, ranging from flatulence, to diarrhea, constipation, and abdominal pain. Other symptoms of gluten sensitivity may include headache, fatigue, skin rash, and peripheral neuropathy, which can have significant effect on the health-related quality of life in patients (Sapone et al., 2012). In addition, while there are no accurate figures available about prevalence, the population affected by NCGS is estimated to be even larger than celiac disease (Jackson et al., 2011). However, the condition remains largely a mystery, with little information available about its mechanism or pathogenic connection to gluten, and there are no serologic markers to aid in the diagnosis of affected individuals.

The information that is logically expected to emerge if the aims of the proposed project are achieved will have the potential to 1) significantly advance our understanding of the relevance of the immune response to gluten in NCGS, and 2) identify potential biomarkers that can be developed further to identify NCGS patients or individuals at risk of developing the condition. As the current state of knowledge of NCGS is highly limited, the proposed experiments are likely to generate substantial preliminary data that will be used effectively to seek external funding from NIH for a series of studies aimed at further research into the mechanism and biomarkers of NCGS.

### Methods

Patients and Controls.

This project will use previously stored (−80° C.) and anonymized specimens. No new patient recruitment would be required, thus eliminating any wait time to receive the samples.

A) NCGS Patients.

Serum samples will be from 20 clinically well-characterized individuals with gastrointestinal complaints and elevated IgG and/or IgA antibodies to gliadin.

B) Celiac Disease Patients.

Serum samples will be from 20 patients with biopsy-proven celiac disease and elevated antibody to gliadin. Diagnosis of celiac disease will have been according to previously described criteria (Alaedini et al., 2005; Briani et al., 2008).

C) Healthy Control Subjects.

In addition to the above disease samples, serum from 40 healthy individuals without any gastrointestinal or other symptoms who are negative for all of the specific celiac disease serologic markers (IgA anti-TG2 antibody, IgG and IgA anti-deamidated gliadin antibody).

Wheat Flour Samples.

Gluten proteins will be extracted from the flour of two different wheat cultivars.

Data Analysis.

All assays will be done in triplicate. Group differences will be analyzed by two-tailed Mann-Whitney U test, Student's t-test, Welch's t-test, or analysis of variance (ANOVA) with post-hoc Dunn test (continuous data), and Chi-square test or Fisher's exact test (nominal data). Cutoff for positivity in all ELISA procedures will be assigned as three standard deviations above the mean for the healthy control group results. Correlation will be measured with Spearman's rank correlation coefficient. Differences with p values of <0.05 will be considered to be significant.

### Construct a Wheat Proteomic Microarray Containing the Full Set of Immunogenic Gluten and Non-Gluten Proteins.

Gluten and non-gluten proteins are extracted from two different wheat varieties, Butte-86 and Cheyenne, as follows. Wheat flour (0.1 mg) is suspended in 1 mL of PBS and mixed for 1 h at 4° C. The mix is centrifuged and the supernatant removed. This fraction contains the water soluble non-gluten albumin/globulin (A/G) proteins. The pellet is treated with 50% isopropanol to extract the gliadin proteins. The remaining pellet after gliadin extraction is resuspended in a solution of 50% isopropanol containing 25 mM DTT and 25 mM Tris-HCl to extract the glutenin proteins. Each collected extract is filtered (0.2 Gliadin and glutenin extracts are combined to form the total gluten extract.

Proteins in the above extracts are separated and fractionated by reversed phase HPLC. 200 uL of extracted protein solution from above is diluted to 1600 uL with 0.1% TFA in water. Injection volumes of 250 uL for the protein solution are separated by gradient elution reversed-phase chromatography using a C18 column. The gradient elution separations are performed at a flow rate of 0.75 mL/min on a Beckman System Gold HPLC system using solvents A=0.1% TFA in water and B=0.08% TFA in acetonitrile. The elution profile is monitored at 214 nm. All fractions are collected at 0.34 min/well in 96-well plates between 5-40 min. In order to demonstrate the feasibility of these fractionations, HPLC separation of the proteins has been attempted. The chromatographic profiles for the gluten and non-gluten proteins of the two wheat varieties showed excellent resolution for the highly complex mixture of the proteins.

The volume of each protein fraction will be adjusted from 200 μL to 50 μL in printing solution (containing 15% glycerol) and spotted on functionalized glass slides at 0.15 nL in triplicate. Each replicate of every array will include human IgG and IgA control spots (0.5-1 ng) for array signal normalization.

### Characterize the Molecular Specificity of the Antibody Response in NCGS.

Antibody Measurement.

Measurement of the level of antibodies to gliadin, glutenin, and non-gluten (A/G) fractions of wheat (Butte-86 and Cheyenne varieties)) will be by ELISA. 96-well round-bottom polystyrene plates (BD Biosciences) will be coated with 50 μL/well of a 0.01 mg/mL solution of extracted proteins from Aim 1 in 0.1 M carbonate buffer (pH 9.6). Control wells will be coated only with buffer. After overnight incubation at 4° C., all wells will be washed and blocked by incubation with 1% BSA in PBST for 1.5 h at room temperature. Serum samples will be diluted at 1:200 for IgA measurement and 1:800 for IgG measurement and added at 50 μL/well in duplicates. After washing the wells, they are incubated with peroxidase-conjugated goat anti-human IgG (Amersham Biosciences) or IgA (MP Biomedicals) secondary antibody for 1 h. Developing solution will be comprised of 27 mM citric acid, 50 mM Na₂HPO₄, 5.5 mM o-phenylenediamine, and 0.01% H₂O₂ (pH 5). Absorbance is measured at 450 nm after incubating the plates for 30 min. Absorbance values will be corrected for non-specific binding by subtraction of the absorbance of the corresponding non-coated wells. Values will be normalized based on mean of two positive controls on each plate. Cutoff values will be assigned as two standard deviations above the mean for the healthy control (negative serology) group results.

Detection of specific celiac disease markers (IgA anti-TG2 antibody, IgG and IgA anti-deamidated gliadin antibody) can be done as previously reported, using prepared kits (Euroimmun) (Samaroo et al., 2010).

Molecular Specificity of Antibody Reactivity.

Each constructed array is blocked with 1% BSA in TBS and incubated with 1:100 dilutions of serum samples in TBST for 2 h. Slides will be washed and incubated with Cy5-labeled anti-human IgG and Cy3-labeled anti-human IgA (Jackson ImmunoResearch) (0.8 μg/mL; 1 h). Arrays will be washed with TBST and de-ionized water, dried under a stream of nitrogen, and scanned using a GenePix 4000B Axon instrument with data acquisition at 570 and 670 nm. The data will be analyzed using the GenePix Pro 6.2 software. Signals for all spots will be normalized based on the control IgG and IgA spot signals on each array. A signal value will be considered positive if it is ≧3 times its respective background signal (SNR≧3) and ≧2 times its standard deviation. Antibody reactivity to spots will be confirmed and further analyzed by ELISA and immunoblotting, as shown (Samaroo et al., 2010; DuPont et al., 2005). Target protein bands of interest will be identified by LC-MS/MS-assisted peptide mass mapping

Anticipated Results and Alternative Approaches.

The proposed experiments will yield a detailed map of the specificity of the antibody response to gluten (and non-gluten) proteins in NCGS. It is expected the antibody response in NCGS to target a unique set of proteins, which would be significantly different from celiac disease. As such, it may offer biomarkers that could be useful for identification and follow-up of NCGS patients or individuals at risk of developing the condition. Furthermore, identification of the target proteins will give novel clues regarding the mechanism of the antibody response in NCGS and its pathogenic relevance. The inclusion of two different wheat cultivars will identify potential differences in reactivity towards the two sets of proteins and serve in confirming the accuracy of the generated data. In addition, the advantage of using the Butte-86 wheat variety is that in a recently completed study from USDA, almost all of the glutens of this cultivar have been identified (Chandra et al., 2011). This database will be extremely useful for peptide mass identification of the target glutens.

Despite the strength of the proposed approach and its potential in deciphering the molecular specificity of the antibody response in NCGS, it suffers from two shortcomings. First, the separated proteins are at different concentrations in each fraction. It is possible that the detection of differential reactivity to proteins that are expressed at very low levels would be missed. This pitfall would also exist if two-dimensional Western blotting (WB) were used. In contrast to WB, however, there will be much smaller intra-assay variation in the microarray approach and the generated data will be substantially more quantitative. In addition, any target fraction on the microarray can be further analyzed by WB to derive more accurate identification of single target proteins, including less abundant molecules. Second, several previous studies have shown that pancreatic and intestinal enzymes are unable to fully digest gluten proteins, resulting in a number of large peptides, some of which are highly immunogenic (Alaedini et al., 2007). Using full length proteins might miss patient antibody responses to certain partially digested peptides of gluten proteins. In order to overcome this pitfall, a pepsin/trypsin/chymotrypsin digest of gluten from both Butte-86 and Cheyenne varieties will be used for construction of the array. 1 mg of the lyophilized total gluten extract from above will be placed in 1 mL of 0.01M HCl and incubated in a 37° C. with pepsin (1:100 protease to protein, Sigma) at pH 2.0 for 30 min. Reaction mixture pH will be adjusted to 7.0 in 50 mM phosphate buffer containing trypsin (1:100, Sigma) and chymotrypsin (1:100, Sigma) and incubated at 37° C. for 2 h. The generated peptides are separated and fractionated by reversed phase HPLC and printed on the same arrays as the proteins, as described above.

### Example 2

### Molecular Characterization of the Immune Response to Gluten in Schizophrenia

Preliminary data in this application demonstrate that the anti-gluten immune response in schizophrenia (SZ) differs significantly from that in celiac disease, displaying a unique antigenic specificity that is independent of the action of transglutaminase enzyme and presentation by HLA-DQ2 and -DQ8 molecules. It is hypothesized that the antibody reactivity to gluten in SZ patients targets a unique set of gluten proteins and epitopes, which can be utilized to understand the disease mechanism and identify novel biomarkers. The present example proposes a systematic approach to assess the relevance of the observed immune response to gluten in SZ patients by fully characterizing its molecular specificity using an gluten microarray system, as follows.

### Characterize the Molecular Specificity of the Anti-Gluten Immune Response in Schizophrenia.

Preliminary work relied on ELISA, size exclusion chromatography, and 1-dimensional immunoblotting to show the presence of a unique immune response to gluten in SZ patients. The proposed experiments in this example will expand the earlier studies through an innovative microarray methodology that will yield accurate data about the molecular specificity of the anti-gluten immune response in SZ. Gluten proteins from two different U.S. wheat cultivars will each be purified into 96 different fractions through two-dimensional chromatography and localized on functionalized glass slides in microarray format. Antibody reactivity to spotted fractions will be detected by using fluorescent-tagged secondary antibodies and an array scanner. Target proteins of interest will be identified by LC-MS/MS-assisted peptide mass mapping. Antigenic determinants of significant target proteins will be determined by epitope mapping.

### Research Strategy

Schizophrenia (SZ) is a chronic, severe, and debilitating mental disorder that affects about 1% of Americans (National Institute of Mental Health, 2011). It exerts a high negative impact on quality of life for patients and their families, and costs tens of billions of dollars in the U.S. alone (McEvoy, J. P. 2007; Knapp et al., 2004). Despite years of study, the mechanism of SZ remains largely unknown. The disease is generally recognized as having a spectrum, with a gradient of clinical phenotypes and possibly varying etiologies. A critical barrier to a better understanding of the condition, accurate diagnosis and follow up of patients, and discovery of more effective therapies has been the lack of specific biomarkers for SZ disease subsets. Immune system abnormalities, including significantly increased antibody response to dietary gluten proteins, have been reported in a substantial number of patients (Mueser et al., 2004). Recently published reports point to increased circulating anti-gluten antibody levels in 20-30% of individuals with SZ (Cascella et al., 2009; Dickerson et al., 2010; Jin et al., 2010). Considering the current absence of and critical need for biomarkers of SZ, the observed increased levels of anti-gluten antibodies in nearly one third of patients is of special significance. It warrants further study to determine the molecular specificity of the antibody response, examine its pathogenic relevance, and assess its potential as a source of biomarkers for identification of patients with shared disease etiopathology.

Preliminary data within this application show that the observed elevated immune response to gluten in SZ is not the same as that in celiac disease (the prototype gluten sensitivity), targeting a unique set of proteins and employing a different mechanism that appears to be independent of antigen presentation by HLA-DQ2/DQ8 molecules or the deamidating activity of TG2 enzyme (Samaroo et al., 2010; Dickerson et al., 2010). It is hypothesized that there is a signature pattern of antibody reactivity directed at specific gluten molecules in individuals with SZ, which may be utilized as disease markers.

The proposed study represents a systematic approach to assess the relevance of the immune response to gluten in a large subset of SZ patients by fully characterizing and mapping its molecular specificity using an innovative gluten microarray system.

Novel Theoretical Concepts.

Preliminary studies point to significant differences in the immune system response of a large subset of SZ patients in comparison to healthy individuals. While the increased immune response to gluten in SZ has been reported in several past studies and discussed for more than two decades, there have been no attempts to examine the molecular specificity and pathogenic relevance of this immune response. It is speculated that further examination of the relevance of gluten to SZ, particularly the target antigen specificity of the immune response to gluten and the assessment of its pathogenic potential will yield novel clues about the disease and may offer biomarkers to identify specific disease subsets.

Novel Methodological Approach.

Preliminary studies relied on enzyme immunoassays, Western blotting, and mass spectrometry to demonstrate the existence of a unique immune response to gluten proteins in a subset of SZ patients. These studies can be expanded through two dimensional fractionation of gluten proteins and the use of an innovative gluten microarray system, an approach that has not been attempted previously. The described experiments are expected to result in a large body of data that can accurately delineate the antigen and epitope specificity of the immune response to gluten in SZ.

### Methodology and Analysis

Patients and Controls.

This project will use previously stored, completely anonymized, specimens.

A) SZ Patients.

Serum samples will be from 30 individuals with multiepisode schizophrenia (including 15 patients with elevated anti-gluten antibody levels and 15 without elevated anti-gluten antibody levels). Patients will be aged between 18 and 45 and diagnosed with schizophrenia meeting criteria in the DSM-IV (Association, 1994).

B) Celiac Disease Patients.

Serum samples will be from 20 patients with celiac disease and elevated antibody to gluten. Diagnosis of celiac disease will have been according to previously described criteria (Alaedini et al., 2005). The samples will be age-matched with those from the SZ patient groups.

C) Healthy Control Subjects.

In addition to the above disease samples, serum from 20 healthy individuals, without celiac disease, neurologic disease or psychiatric symptoms (as confirmed by clinical examination and screening with the Structured Clinical Interview for DSM-IV Axis I Disorders—Nonpatient Edition (First et al., 1998) will be included. The samples will be age-matched with those from the SZ patient groups.

Data Analysis.

All assays will be done in triplicate. Group differences will be analyzed by two-tailed Mann-Whitney U test, Student's t-test, or Welch's t-test, or analysis of variance (ANCOVA) with post-hoc Dunn test (continuous data), and Chi-square test or Fisher's exact test (nominal data). Cutoff for positivity in all ELISA procedures will be assigned as three standard deviations above the mean for the healthy control group results. Correlation will be measured with Spearman's rank correlation coefficient. Differences with p values of <0.05 will be considered to be significant.

### Characterize the Molecular Specificity of the Anti-Gluten Immune Response in Schizophrenia.

Antibody Detection.

Measurement of levels of antibodies to gluten will be by ELISA as previously described (Samaroo et al., 2010).

Molecular Specificity of Anti-Gluten Antibody Reactivity.

Gluten will be extracted and separated into 6 fractions by size exclusion chromatography as previously described (Samaroo et al., 2010). Fractions are dissolved in 6 M guanidine HCl (pH 8.0), containing 50 mM DTT. 500 μL aliquots are applied to a C18 semipreparative RP-HPLC column and eluted using a Varian Prostar system and gradient elution with water:acetonitrile:TFA (DuPont, 2005). The elution profile is monitored at 210 nm and individual peaks are collected and lyophilized. Each of the 6 fractions will be separated further into 15-20 peaks, for a total of approximately 100 fractions, each representing 1-3 unique gluten proteins. Each eluted fraction will be spotted on functionalized glass slides in triplicate using a Molecular Dynamics printer (GE). Arrays will be blocked, incubated with patient sample, processed, and the data analyzed as recently described in detail (Chandra et al., 2011). Antibody reactivity to spots will be confirmed by ELISA and immunoblotting as shown (Samaroo et al, 2010; Chandra, 2011). Target protein bands of interest will be identified by LC-MS/MS-assisted peptide mass mapping as shown (Samaroo et al, 2010; Alaedini et al., 2007).

Epitope Mapping.

In order to determine the linear epitopes of specific target proteins involved in the anti-gluten immune response in SZ, the binding of serum antibodies from selected patients to arrays of overlapping peptides of the reactive protein(s) of interest can be examined. The designed peptides (14mers, with an overlap of 9 amino acids each, based on the sequences of the identified gluten proteins) will be dissolved in printing solution and deposited onto epoxy-functionalized glass slides (Corning). The entire procedure for epitope mapping, including peptide synthesis, array processing, and data analysis can be done as described (Chandra et al., 2011).

Anticipated Results and Alternative Approaches.

The proposed experiments will yield a highly detailed map of the specificity of the anti-gluten antibody response in SZ. It is expected the antibody response in SZ to target a unique set of proteins, which would be significantly different from celiac disease. As such, it has the potential to offer biomarkers that could be useful for identification and follow-up of specific subsets of SZ patients or individuals at risk of developing SZ. Furthermore, identification of the target proteins/epitopes will give novel clues regarding the mechanism of the anti-gluten immune response in SZ and its pathogenic relevance.

### Example 3

### Gluten and Autism

In this study, markers of celiac disease and gluten sensitivity in cohorts of individuals diagnosed with autism, unaffected siblings of the patients with autism, and unrelated healthy controls were examined and compared.

### Patients and Controls

The study included 140 children, including 37 with autism, 27 unaffected siblings of similar ages within the same families, and 76 unrelated healthy controls. Serum samples from individuals with autism and their siblings were acquired from the Autism Genetic Resource Exchange (AGRE). DNA samples from the 37 children with autism were also provided by AGRE. Participants in the AGRE program have been recruited primarily from the north-eastern and western United States. Affected children met the diagnostic criteria for autism based on both the Autism Diagnostic Observation Schedule (ADOS) and the Autism Diagnostic Interview, Revised (ADI-R). All available serum samples satisfying the above criteria were included. Information on GI symptoms was based on parent questionnaires, interviews, and medical histories. The data collected by AGRE from these evaluations were retrieved from the online AGRE phenotype database. The control sera were from healthy children in the United States (n=14) and Sweden (n=62). The healthy controls from U.S. resided primarily in Connecticut, north New Jersey, and New York City, and were recruited in a general pediatric clinic at the Weill Cornell Medical College. The healthy controls from Sweden were recruited at child health care centres and schools in the Falun region of central Sweden (Aldrimer et al., 2012). Screening questionnaires were used to evaluate the general health of the U.S. and Swedish controls, and individuals who reported having a chronic disease were not included. Serum from a biopsy-proven celiac disease patient, diagnosed according to previously described criteria (Alaedini et al., 2005) at Columbia University Medical Center, was used as a positive control for the antibody assays. Written informed consent was obtained for all study participants from the individual, next of kin, caretaker, or guardian. The consent procedures were approved by the Institutional Review Boards of the involved organizations (AGRE, Columbia University, Weill Cornell Medical College, and Uppsala University). Complete documentation of consent is maintained at the respective organizations. This specific study was approved by the Institutional Review Board of Columbia University Medical Center. Specimens were kept at −80° C. to maintain stability.

### Gliadin

The antigen mixture used for the anti-gliadin antibody assays was the Prolamine Working Group (PWG) reference gliadin, which was extracted from a combination of 28 different wheat varieties, as previously described (van Eckert et al., 2006). The protein profile of the PWG gliadin extract was assessed by SDS-polyacrylamide gel electrophoresis, using 10% NuPAGE Bis-Tris precast gels and 3-(N-morpholino)propanesulfonic acid (MOPS) buffer (Life Tech-nologies, Carlsbad, Calif.).

### Anti-Gliadin Antibodies

Serum IgG and IgA antibodies to gliadin were measured separately by enzyme-linked immunosorbent assay (ELISA) as previously described (Alaedini et al., 2007; Samaroo et al., 2010), with some modifications. A 2 mg/mL stock solution of the PWG gliadin was prepared in 60% ethanol. 96-well Maxisorp round-bottom polystyrene plates (Nunc, Roskilde, Denmark) were coated with 50 uL/well of a 0.01 mg/mL solution of PWG gliadin in 0.1 M carbonate buffer (pH 9.6) or were left uncoated to serve as control wells. After incubation at 37° C. for 1 h, all wells were washed and blocked by incubation with 1% bovine serum albumin (BSA) in phosphate buffered saline containing 0.05% Tween-20 (PBST) for 1.5 h at room temperature. Serum samples were diluted at 1:800 for IgG measurement and at 1:200 for IgA measurement, added at 50 uL/well in duplicates, and incubated for 1 h. Each plate contained a positive control sample from a patient with biopsy-proven celiac disease and elevated IgG and IgA antibodies to gliadin. After washing the wells, they were incubated with HRP-conjugated anti-human IgG (GE Healthcare, Piscataway, N.J.) or IgA (MP Biomedicals, Santa Ana, Calif.) secondary antibodies for 50 min. The plates were washed and 50 uL of developing solution, comprising of 27 mM citric acid, 50 mM Na₂HPO₄, 5.5 mM o-phenylenediamine, and 0.01% H₂O₂ (pH 5), was added to each well. After incubating the plates at room temperature for 20 min, absorbance was measured at 450 nm. All serum samples were tested in duplicate. Absorbance values were corrected for non-specific binding by subtraction of the mean absorbance of the associated BSA-coated wells. The corrected values were first normalized according to the mean value of the positive control duplicate on each plate. The mean antibody level for the unrelated healthy control cohort was then set as 1.0 AU and all other results were normalized accordingly.

### Anti-Transglutaminase 2 (TG2) Antibodies

IgA antibody to recombinant human TG2 was measured in sera using an ELISA kit, according to the manufacturer's protocol (Euroimmun, Lubeck, Germany).

### Anti-Deamidated Gliadin Antibodies

Sera were tested separately for IgG and IgA antibodies to a previously described glutamine-glutamate substituted trimer of a fusion peptide containing the sequences PLQPEQPFP and PEQLPQFEE (Schwartz et al., 2004) by ELISA, according to the manufacturer's protocols (Euroimmun).

### HLA Typing

High resolution HLA genotyping was performed by multiplex polymerase chain reaction (PCR) with biotinylated primers, followed by reverse hybridization of the PCR products to line arrays of sequence-specific DQA1 and DQB1 oligonucleotide probes, using INNO-LiPA HLA-DQ kits, according to the manufacturer's instructions (Innogenetics, Gent, Belgium). Presence or absence of celiac disease-associated DQA1*0501/0505-DQB1*0201/0202 (DQ2) and DQA1*03-DQB1*0302 (DQ8) genes was determined.

### Data Analysis

Differences between groups were analyzed by the two-tailed Student's t test, Welch's t test, Mann-Whitney U test, or one-way analysis of variance (ANOVA) with post-hoc Dunn test (continuous data), and the Fisher's exact test (nominal data). Adjustment for covariate effect (age, gender, and race) was carried out by analysis of covariance (ANCOVA), using the general linear model. Logistic regression was used to calculate the odds ratios associated with increased antibodies in individuals with autism. For these analyses, increased levels of anti-gliadin antibody were defined as values at the 95th percentile or higher in the unrelated healthy control group. For IgA anti-TG2 antibody and IgG/IgA anti-deamidated gliadin antibodies, cutoffs for positivity were assigned by the manufacturer. Differences with p values of <0.05 were considered to be statistically significant. Statistical analyses were performed with Prism 5 (GraphPad, San Diego, Calif.) and Minitab 16 (Minitab, State College, Pa.).

### Results

The demographic and clinical characteristics of the patients with autism, their unaffected siblings, and unrelated healthy controls are shown in Table 1. The patient cohort included four individuals on gluten-free diet. Because the effect of gluten-free diet on antibody levels in autism is not known, these patients were not excluded from the study.

TABLE 1
Demographic Characteristics of Study Cohorts

| | Number of | Mean age | Male sex | White race |
|---|---|---|---|---|
| Subject group | subjects | years ± SD | no.(%) | no.(%) |
| Autism | 37 | 7.8 ± 2.9 | 29 (78) | 33 (89) |
| With GI symptoms | 19 | 7.1 ± 2.3 | 13 (68) | 15 (79) |
| Without GI symptoms | 8 | 7.1 ± 2.3 | 6 (75) | 8 (100) |
| Unaffected sibling | 27 | 8.1 ± 2.9 | 18 (67) | 25 (93) |
| Unrelated healthy | 76 | 8.8 ± 3.7 | 59 (77) | 70 (92) |

### Gliadin

The gel electrophoresis profile for the PWG gliadin used in anti-gliadin antibody assays indicated the presence of all main types of gliadin proteins, α/β, γ, and ω. The mixture also contained high and low molecular weight glutenin subunits.

### Antibody Levels

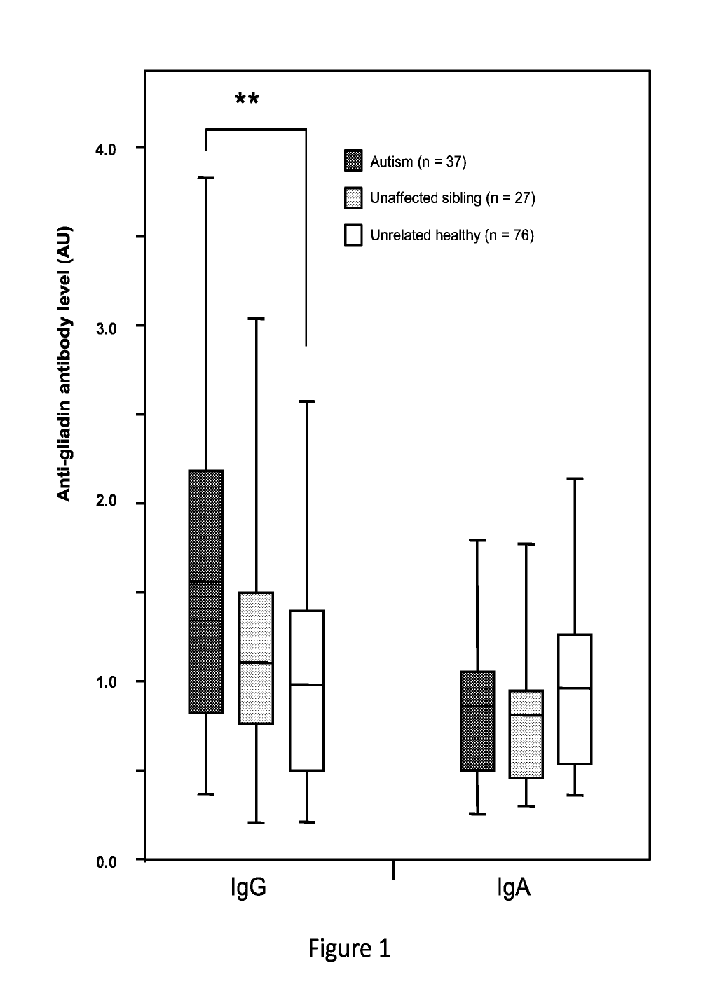

Mean levels of IgG and IgA class antibodies to gliadin in patient and control groups are presented in FIG. 1. Children with autism exhibited significantly elevated levels of IgG antibody to gliadin when compared with unrelated healthy controls or when compared with the combination of unaffected siblings and unrelated healthy controls (p<0.01). The difference remained significant after adjusting for the covariates of age, gender, and race (p<0.01). The anti-gliadin IgG differences between the children with autism and their unaffected siblings, and between the siblings and unrelated healthy controls, did not reach statistical significance. Based on the stated cutoff for positivity (95th percentile of the healthy control group), 8/33 (24.2%) of the children with autism, excluding those who reported being on gluten-free diet, 8/37 (21.6%) of all autistic children, including those on gluten-free diet, 2/27 (7.4%) of unaffected siblings, and 4/76 (5.3%) of unrelated healthy children were positive for IgG anti-gliadin antibody, indicating a significantly higher frequency in those with autism compared to unrelated healthy controls (p<0.01). Children with autism had increased odds of having elevated IgG antibody to gliadin in comparison to healthy controls (odds ratio: 4.97; 95% confidence interval: 1.39-17.8). The differences in levels of IgA antibody to gliadin among the three groups were not significant.

All patients and controls were also tested for the currently recommended full panel of the most sensitive and specific serologic markers of celiac disease, including IgA antibody to TG2, IgG antibody to deamidated gliadin, and IgA antibody to deamidated gliadin. None of the individuals in any group were positive for IgA antibody to TG2. Two of 37 autistic children, 3 of 27 unaffected siblings, and none of 76 unrelated healthy controls had values above the manufacturer's assigned cutoff for IgG antibody to deamidated gliadin. Similarly, none of 37 autistic children, 1 of 27 unaffected siblings, and 1 of 76 unrelated healthy controls were positive for IgA antibody to deamidated gliadin. All four individuals who were on gluten-free diet were negative for anti-gliadin, anti-deamidated gliadin, and anti-TG2 antibodies.

### HLA Typing

In the group of children with autism, 18/37 (48.6%) were positive for HLA-DQ2 and/or -DQ8 (6 DQ2, 12 DQ8). There was no clear association between antibody to gliadin and the presence of celiac disease-associated HLA-DQ2/DQ8 in patients with autism: 3/8 (37.5%) of the anti-gliadin antibody-positive individuals with autism displayed HLA-DQ2 and/or DQ8 (2 DQ2, 1 DQ8), while 15/29 (51.7%) of those below the cutoff for antibody positivity had DQ2 and/or DQ8. Neither of the two patients with autism who were positive for IgG anti-deamidated gliadin antibody had DQ2 or DQ8. About 95% or more of celiac disease patients carry HLA-DQ2 and/or -DQ8, compared to an estimated 40% of the U.S. general population (Kagnoff, 2007).

### GI Symptoms

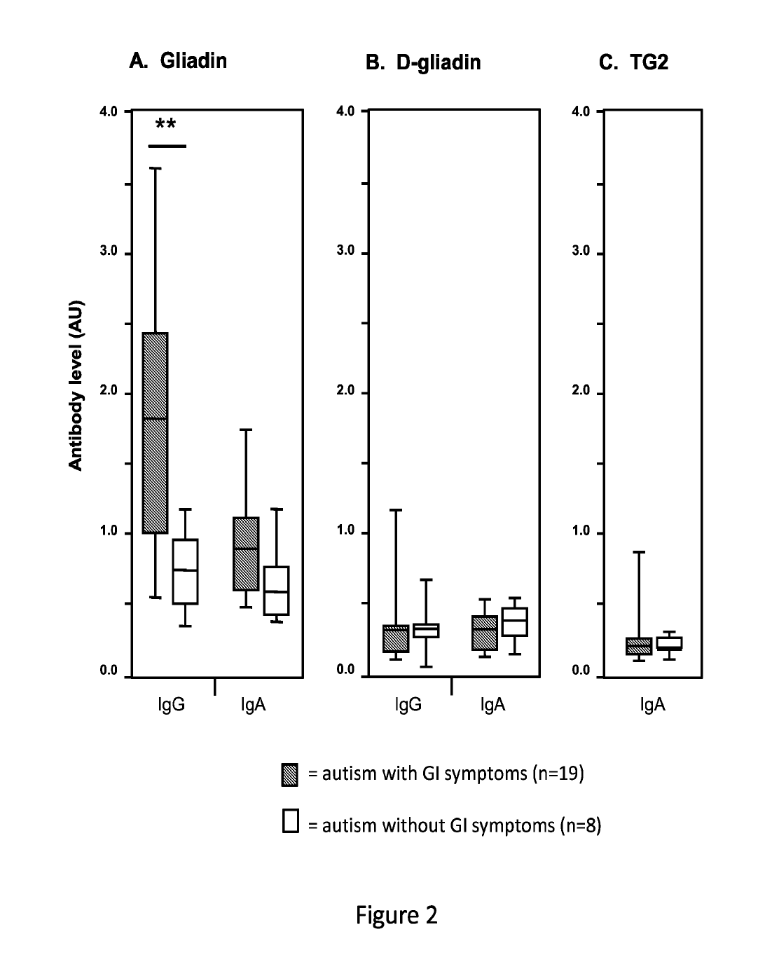

Medical histories were available for 27 of the 37 children with autism. 19/27 (70.3%) reported persistent GI symptoms, including 10 with chronic loose stools or diarrhea, 2 with gastroesophageal reflux, 3 with frequent stools, 3 with constipation, and 1 with non-specified GI symptoms. Affected patients with GI symptoms were found to have significantly higher levels of IgG antibody to gliadin when compared to patients without GI symptoms (p<0.01) (FIG. 2A). This difference remained significant after adjusting for the covariates of age, gender, and race (p<0.01). Information on GI symptoms was available for 5 of the 8 children whose anti-gliadin antibody levels were determined to be above the cutoff. They included 3 with chronic loose stools or diarrhea, 1 with frequent stools, and 1 with constipation.

There was no significant difference in the levels of IgA antibody to gliadin (FIG. 2A), IgG and IgA antibodies to deamidated gliadin (FIG. 2B), and IgA antibody to TG2 (FIG. 2C) between patients with GI complaints and those without. One autism patient with GI symptoms was positive for IgG antibody to deamidated gliadin, while the remaining patients in both groups were negative for all other markers.

### Discussion

The aim of this study was to carry out a comprehensive analysis of markers of celiac disease and gluten sensitivity in a group of children with autism who had been diagnosed according to strict criteria and defined instruments. The data indicate that children with autism have higher levels of IgG antibody to gliadin compared to healthy controls. In addition, among patients with autism, the antibody response to gliadin was greater in those with GI symptoms. However, in contrast to patients with celiac disease, no association was observed between the elevated anti-gliadin antibody level and the presence of highly specific serologic markers of celiac disease or HLA-DQ2/DQ8. The findings indicate that the observed anti-gliadin immune response in patients with autism is likely to involve a mechanism that is distinct from celiac disease, without the requirement for TG2 activity or antigen presentation through DQ2/DQ8 MHC molecules (Alaedini, 2008).

The data from this study should be interpreted with caution. Most importantly, the observed increased IgG antibody response to gliadin does not necessarily indicate sensitivity to gluten or any pathogenic role for antibodies to gliadin in the context of autism. In addition, the results do not rule out the possibility of moderately increased prevalence of celiac disease among children with autism, especially as duodenal biopsy, the gold standard for definitive diagnosis of celiac disease, was not performed. However, considering the excellent sensitivity and specificity of anti-TG2 and (and to a lesser extent anti-deamidated gliadin) antibodies, as well as the high negative predictive value of HLA-DQ2/DQ8 markers for celiac disease, it can be concluded with high certainty that the overwhelming majority of autism patients with elevated antibody to gliadin do not have celiac disease. If future studies prove the existence of sensitivity to gluten in a subset of patients with autism, the gluten-associated symptoms in such individuals may fall within the spectrum of “non-celiac gluten sensitivity” (Lundkin, 2012).

Compared to previous reports examining the link between celiac disease/gluten sensitivity and autism, this study is unique in several ways. First, a shortcoming in earlier studies has been the lack or incompleteness of suitable age-matched healthy control groups necessary for this type of analysis. In this work, the antibody levels in children with autism were compared to two separate pediatric control groups: unaffected siblings of the same patients, as well as a larger cohort of unrelated healthy children. Second, previous reports have used specimens from more heterogeneous groups of patients generally recruited at local hospitals or clinics, and while most report the use of DSM diagnostic criteria, it is unclear which test(s) informed the final diagnosis of autism. In contrast, the samples in this study were acquired from a well-recognized repository of biomaterials (AGRE), which is managed by the world's largest autism advocacy organization and has been utilized in various past research projects. The associated AGRE database includes information about family pedigree, scores from various tests and questionnaires, and medical histories for many of the patients for which biospecimens are available. Patients in this study were selected only if they were identified as having autism according to two separate instruments, ADOS and ADI-R, thus greatly increasing the likelihood of accurate diagnosis.

A limitation of this study is that geographical distribution, socioeconomic status, or diet of the research participants were not controlled for. These factors may contribute to levels of antibodies against dietary and other antigens in patients and controls. In addition, information on GI symptoms was available only for some patients and none of the controls. Access to such data would have strengthened the study's finding regarding the association between GI symptoms and anti-gliadin antibody levels.

There are several possibilities to explain the higher anti-gliadin antibody levels found in the cohort of children with autism. Previously, associations between autism and increased GI symptoms, as well as impaired intestinal permeability, have been reported (D'Eufemia, 1969; de Magistris et al., 2010; Brown et al, 2011). Increased intestinal permeability resulting from damage to the intestinal epithelial barrier in those with autism may be responsible for increased exposure of the immune system to partially digested gluten fragments, resulting in the detected increase in antibody response. The observation here that anti-gliadin antibody reactivity is elevated in patients with GI symptoms lends some support for this idea. At the same time, the fact that the higher anti-gliadin antibodies in autistic children were limited to the IgG isotype, without a concomitant rise in IgA, may imply a non-mucosal and/or gluten-independent origin for the observed antibody reactivity. One possibility is that the IgG-specific antibody response in children with autism would have been triggered by ingested gluten at some point in the past, but no longer dependent on continuous mucosal exposure to the proteins. Alternatively, the detected anti-gliadin antibodies may be unrelated to gluten as the immunogen. Various immune abnormalities have been demonstrated in autistic children, including increased antibody reactivity to autoantigens (Frye et al., 2012; Goines et al., 2011; Zhang et al., 2010). It is conceivable that certain autism-associated autoantibodies, the exact targets of which are yet to be identified, would cross-react with one or more gluten proteins and contribute to the detected difference in anti-gliadin antibody level between patients and controls. Circulation levels of such antigen-independent or gluten cross-reactive antibodies would not be expected to respond to dietary gluten restriction.

Results of this study are intriguing in the context of disease pathophysiology and biomarker identification. The observed increase in antibody reactivity to gliadin in over one fifth of the autism cohort points to potential shared genetic and/or environmental associations in a sizable subset of patients. As such, the generated data provide an impetus to further examine the affected patient subset for additional immunologic and genomic clues. It is possible that, in a subset of children with autism, the condition is associated with antibody reactivity to a unique set of gluten proteins that would be significantly different from the pattern of anti-gliadin antibody response in celiac disease and other conditions. This specific pattern of antibody reactivity may be useful as a source of biomarkers. A unique antibody response to particular gluten molecules could also be associated with specific HLA genes in that disease subset.

In conclusion, the increased anti-gliadin antibody response in autism and its association with GI symptoms points to a potential mechanism involving immunologic and/or intestinal permeability abnormalities in a subset of patients. The observed antibody reactivity to gliadin in most children with autism appears to be unrelated to celiac disease. Therefore, the heightened immune response to gluten in autism deserves further attention and research in determining its utility as a source of biomarkers and clues regarding disease pathophysiology. Better understanding of this immune response may offer novel markers for the identification of subsets of patients who would be responsive to specific treatment strategies. The wheat proteomic array invention disclosed herein can be used for deciphering the target specificity of the anti-gluten antibody response in the affected patients.

### Example 4

### Gluten Microarray

### Progress in Construction of the Wheat Proteomic Array.

Array Protein and Peptide Content.

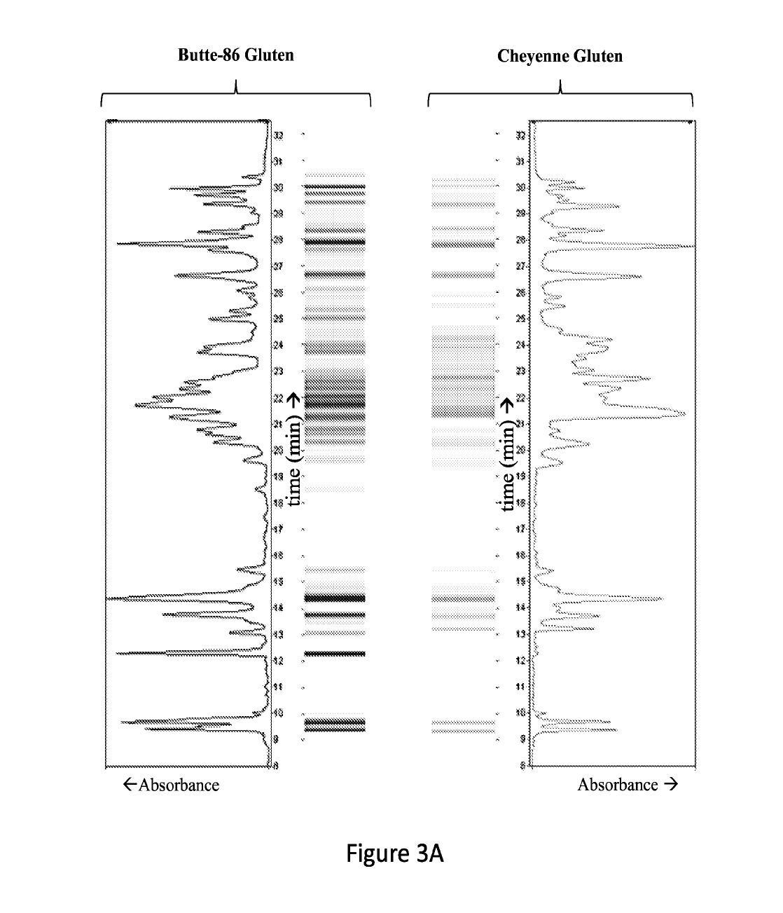

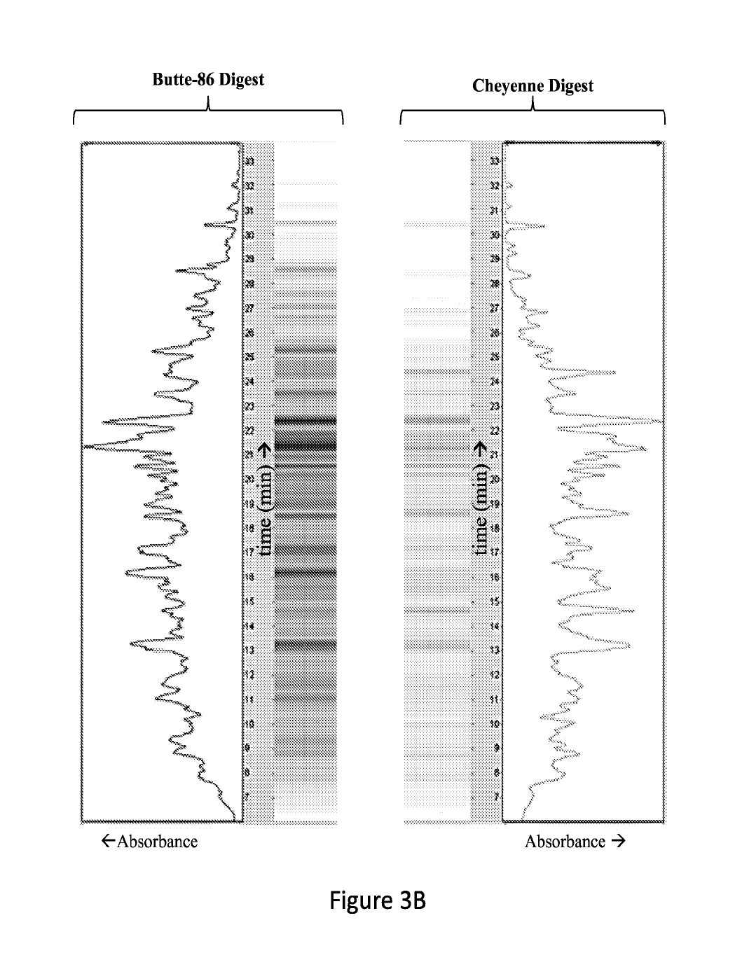

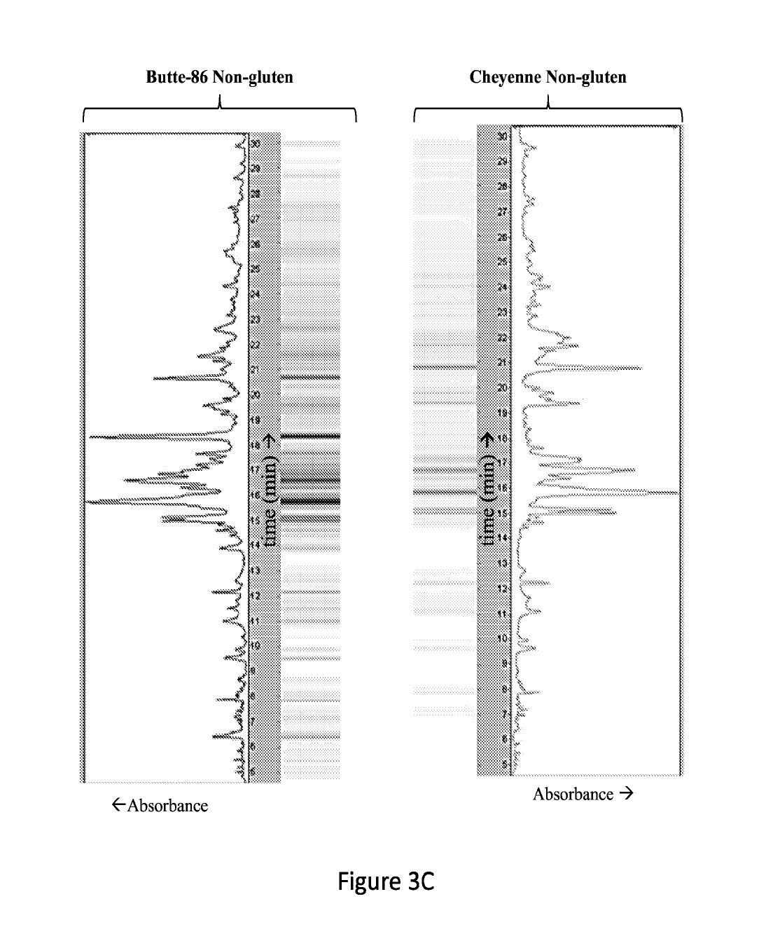

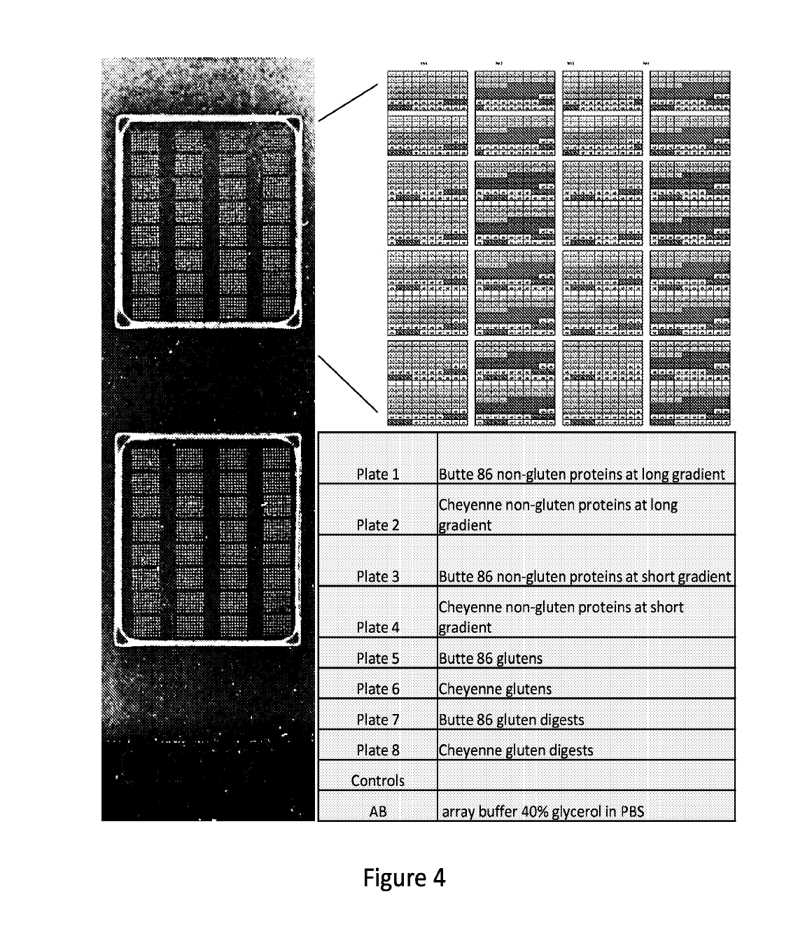

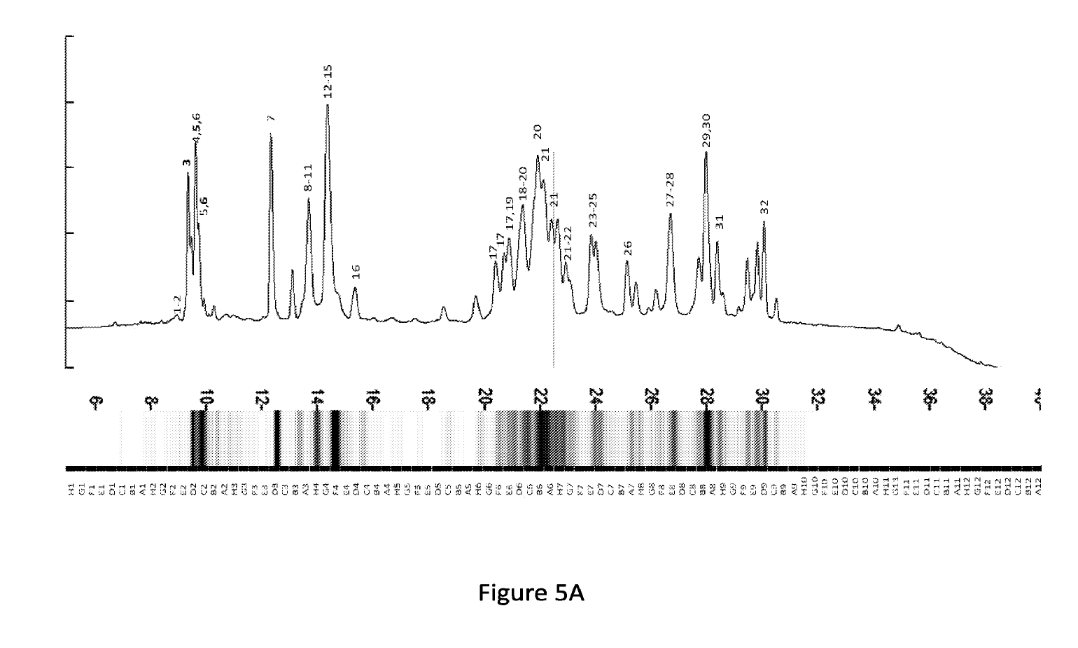

In addition to the previously described inclusion of wheat gluten and non-gluten fractions on the array, the constructed array now also contains HPLC-separated fractions from an enzymatic digest of wheat gluten proteins (thus simulating the natural process of digestion). FIG. 3 shows the chromatographic separation of the various wheat extractions, as well as the digests. FIG. 4 shows the location of the printed fractions on the arrays.

Identification of HPLC Fraction Protein Contents.

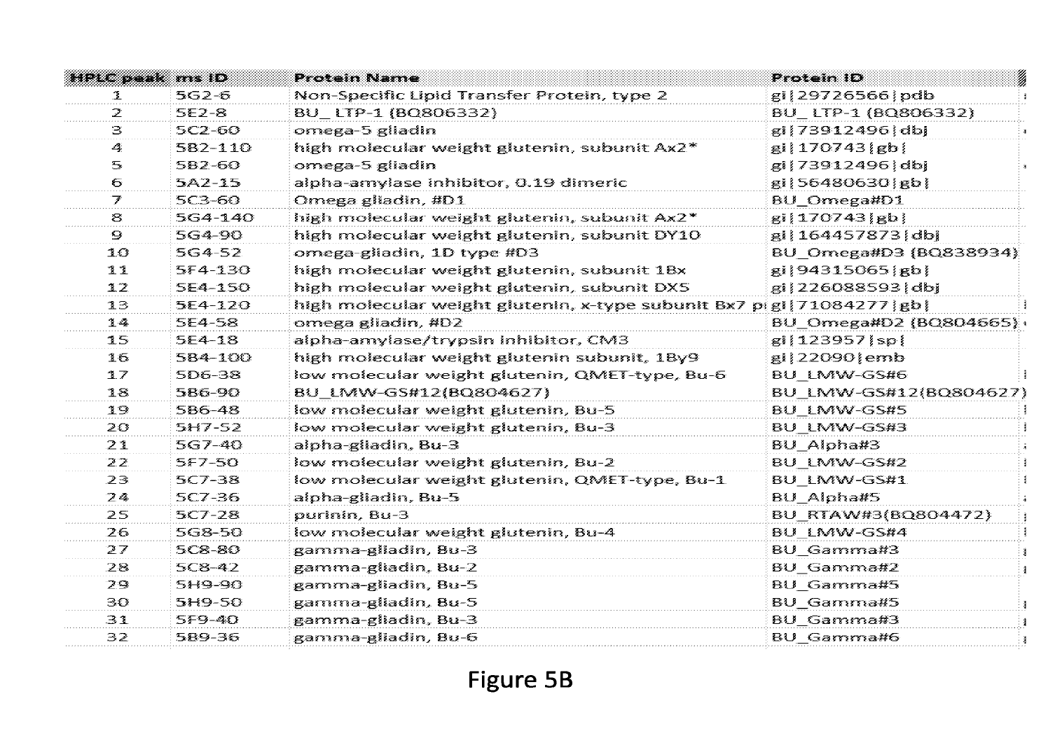

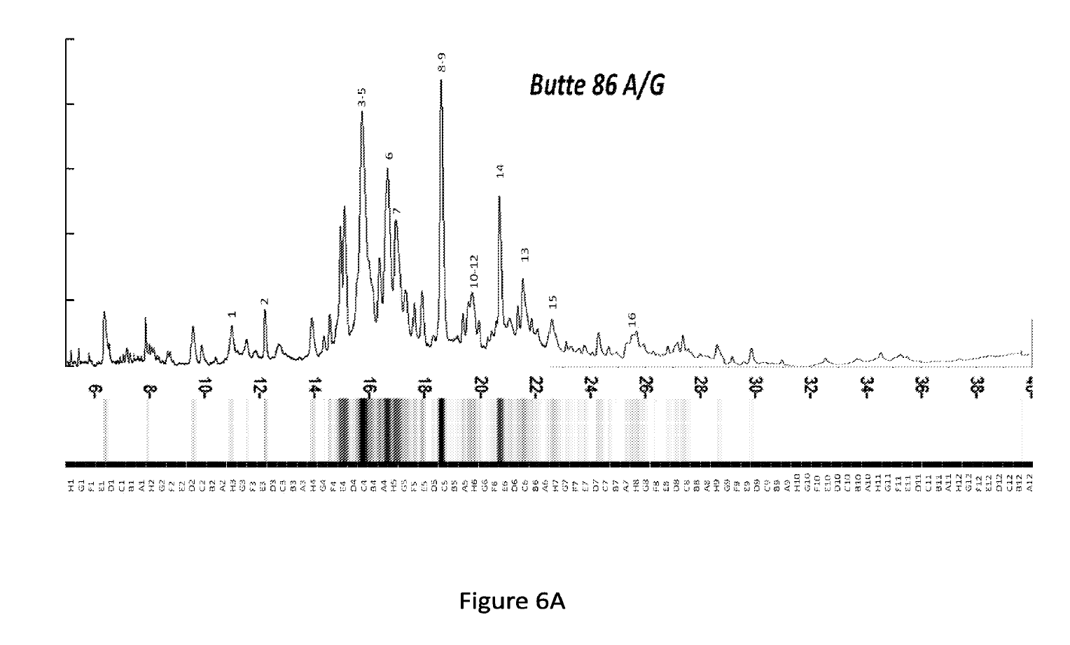

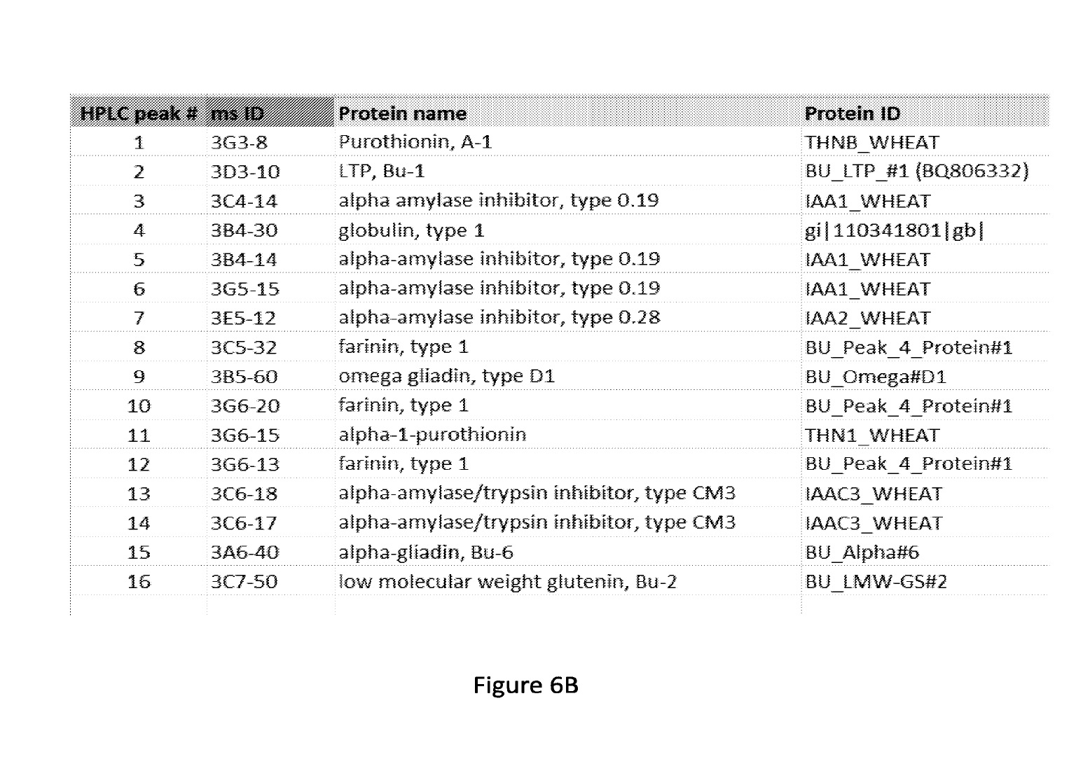

The major gluten and non-gluten protein components of the chromatographic fractions have been identified by liquid chromatography-tandem mass spectrometry (LC-MS/MS)-assisted peptide mass mapping as previously described (Samaroo et al., 2010). FIGS. 5 and 6 demonstrate the identities of constituent proteins in major peaks.

Assessment of Reactivity of Serum Antibodies from Representative Patients Using the Microarray.

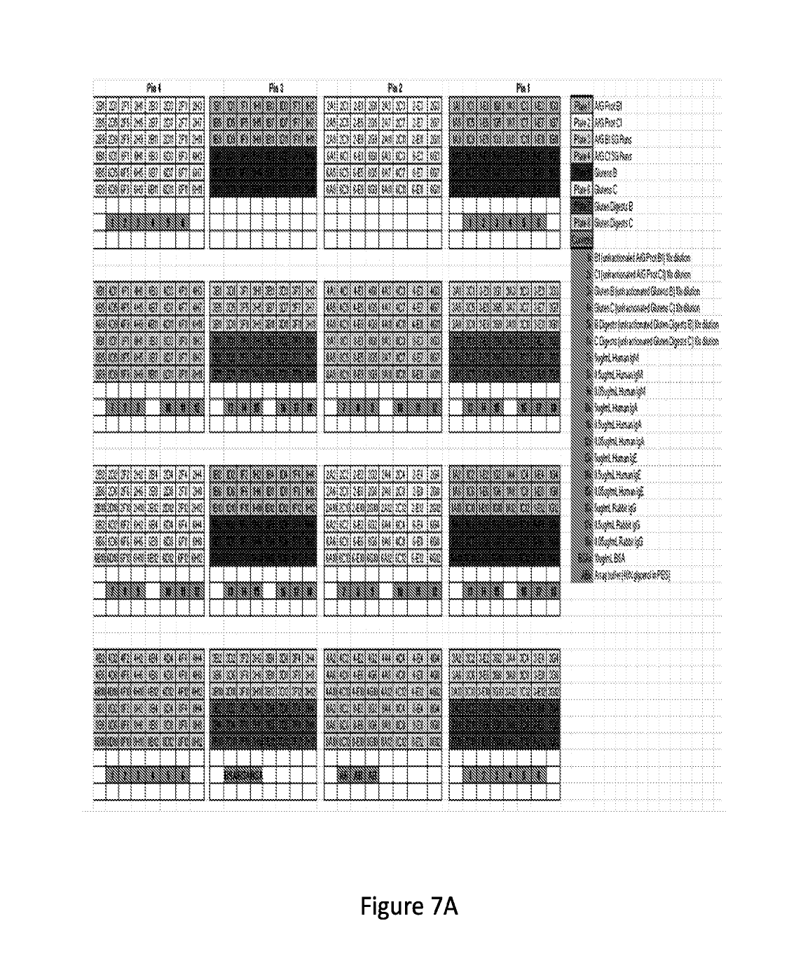

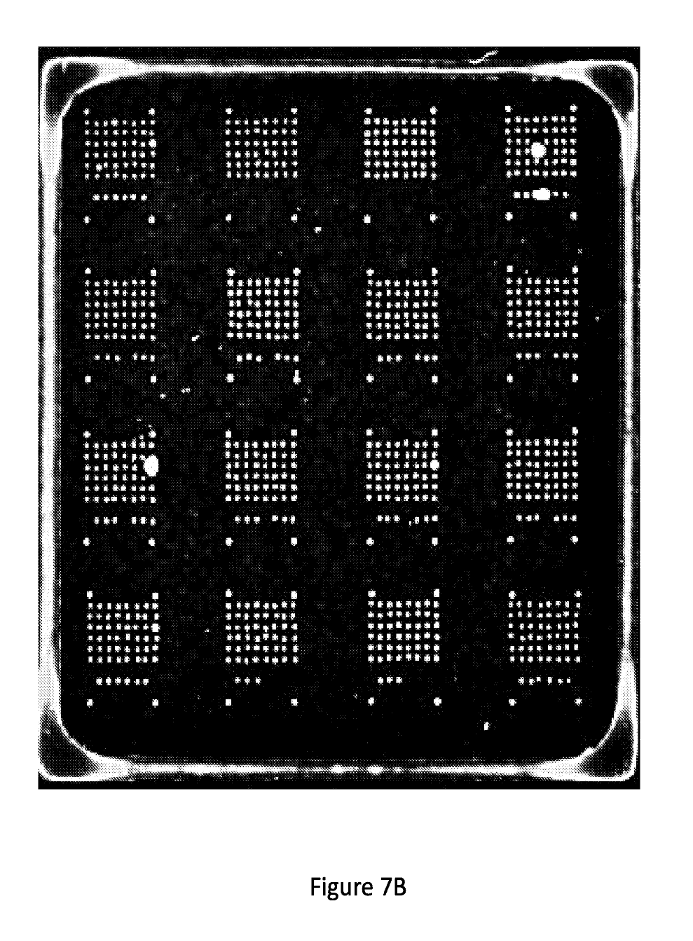

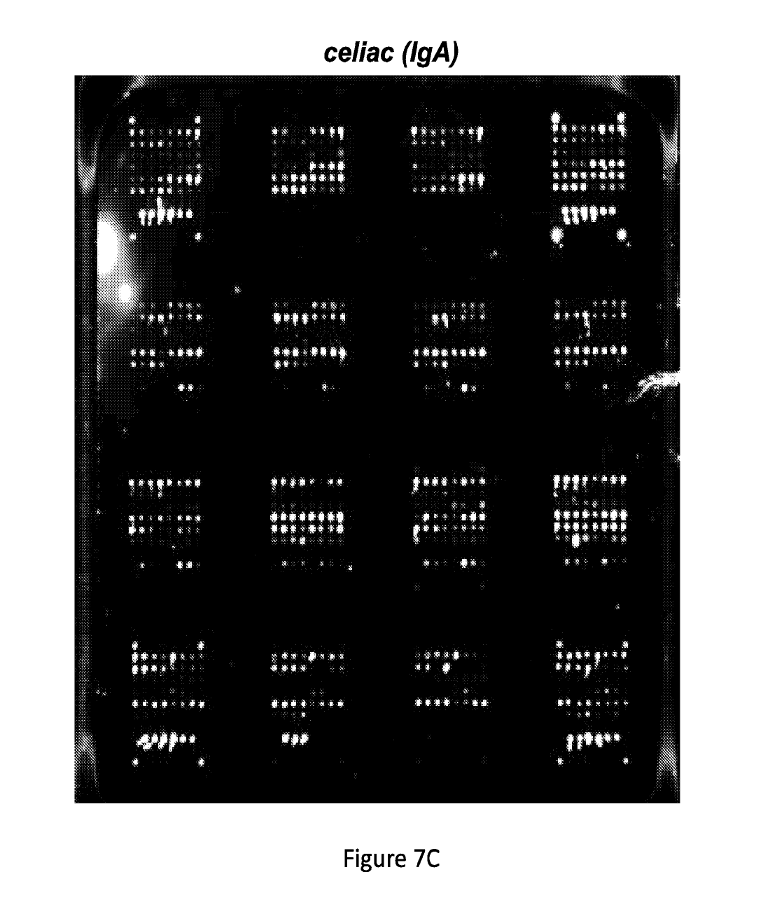

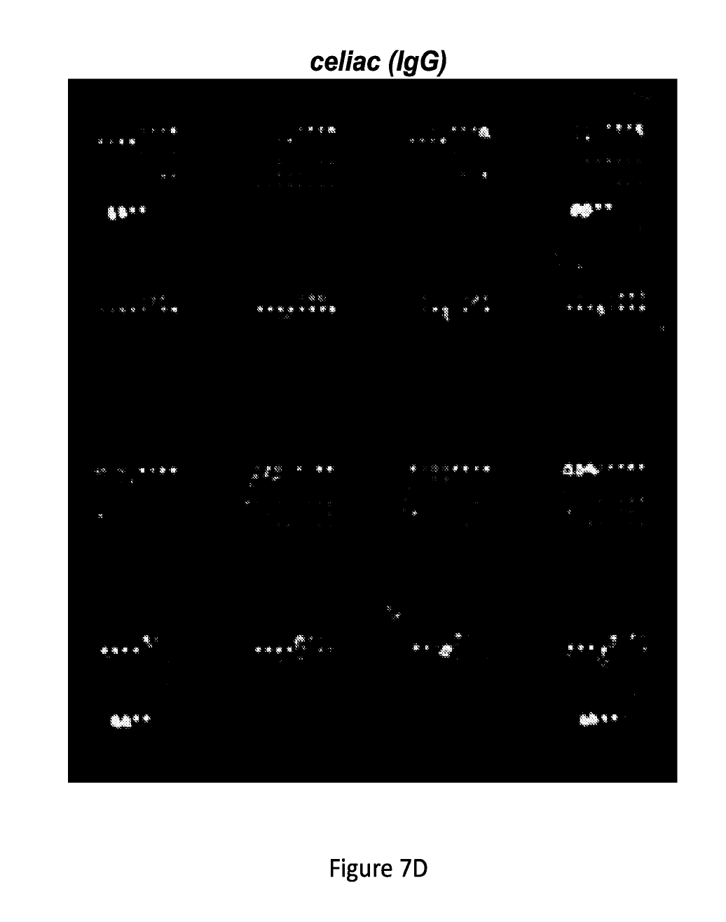

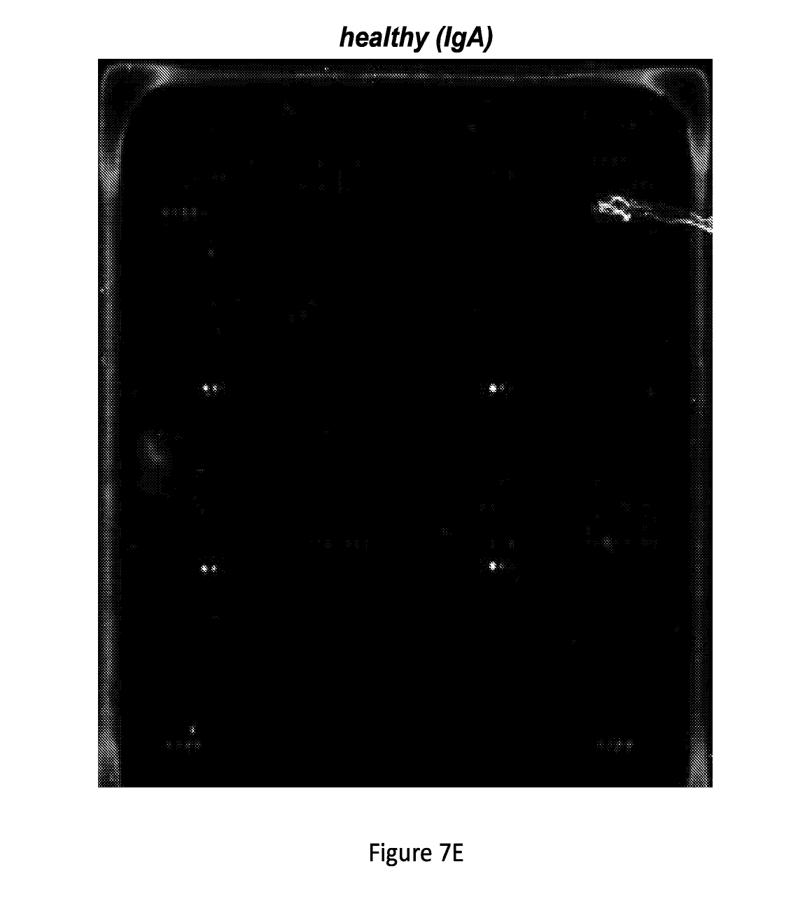

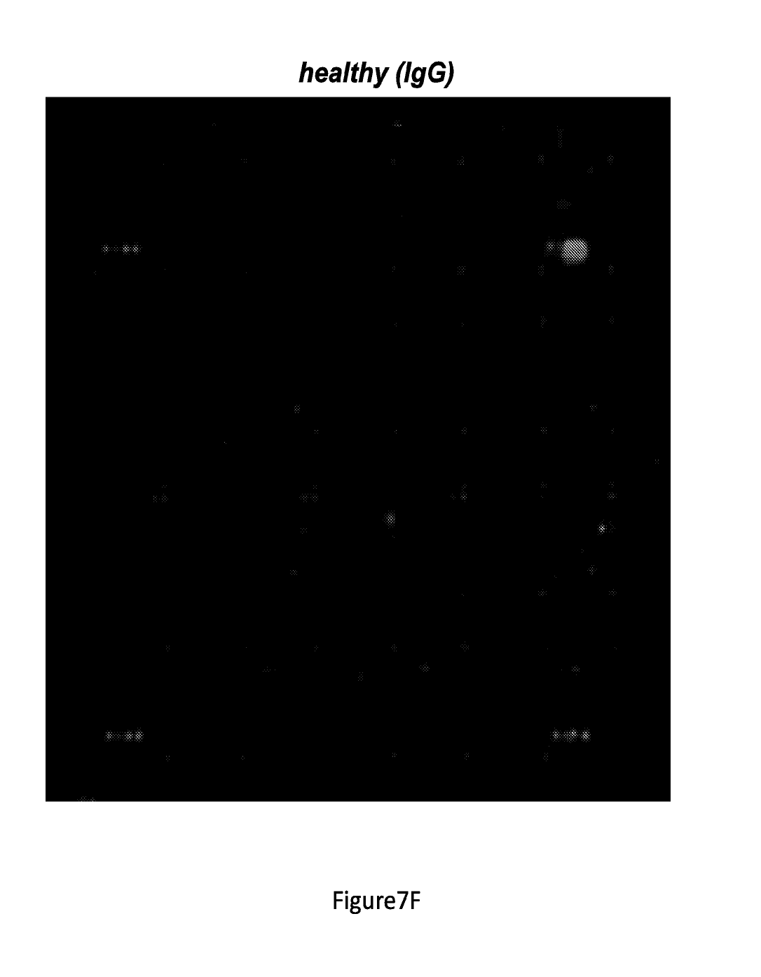

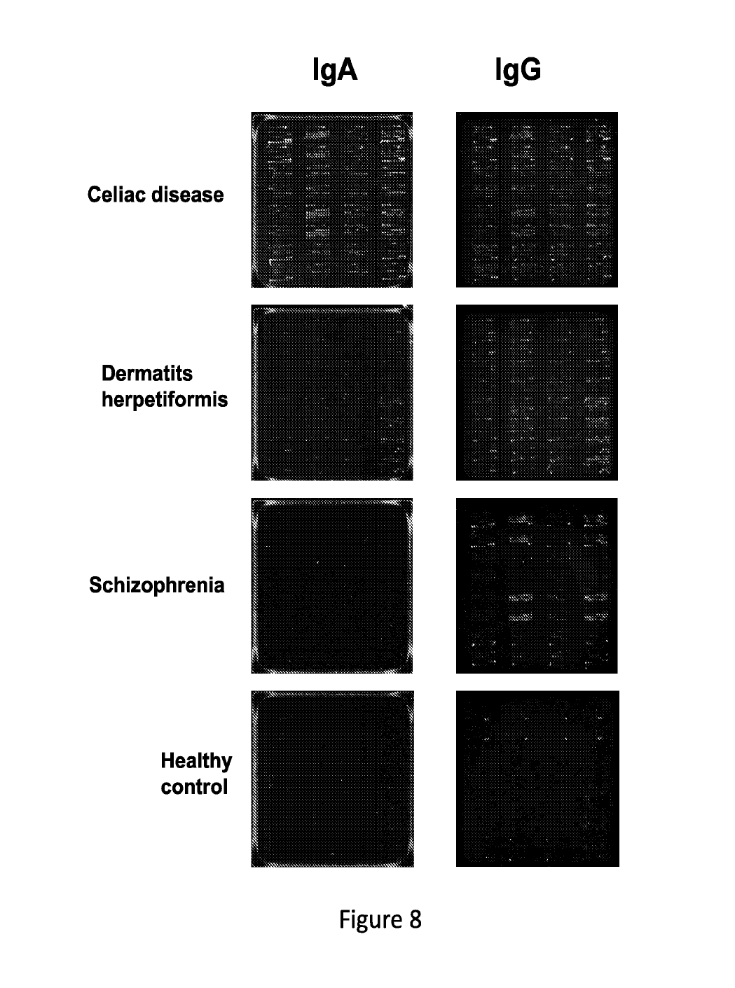

There are preliminary data demonstrating the utility and power of the constructed array. FIG. 7 shows images from the microarray prototype, demonstrating IgG and IgA antibody analyses for a celiac disease patient and a healthy individual. FIG. 8 shows the images of array immune reactivity for 4 representative subjects with celiac disease, dermatitis herpetiformis, and schizophrenia, as well as a healthy individual. Arrays were scanned with a GenePix 4000B Axon instrument for simultaneous acquisition of IgG and IgA antibody data. Data were analyzed using the GenePix Pro 6.0 software.

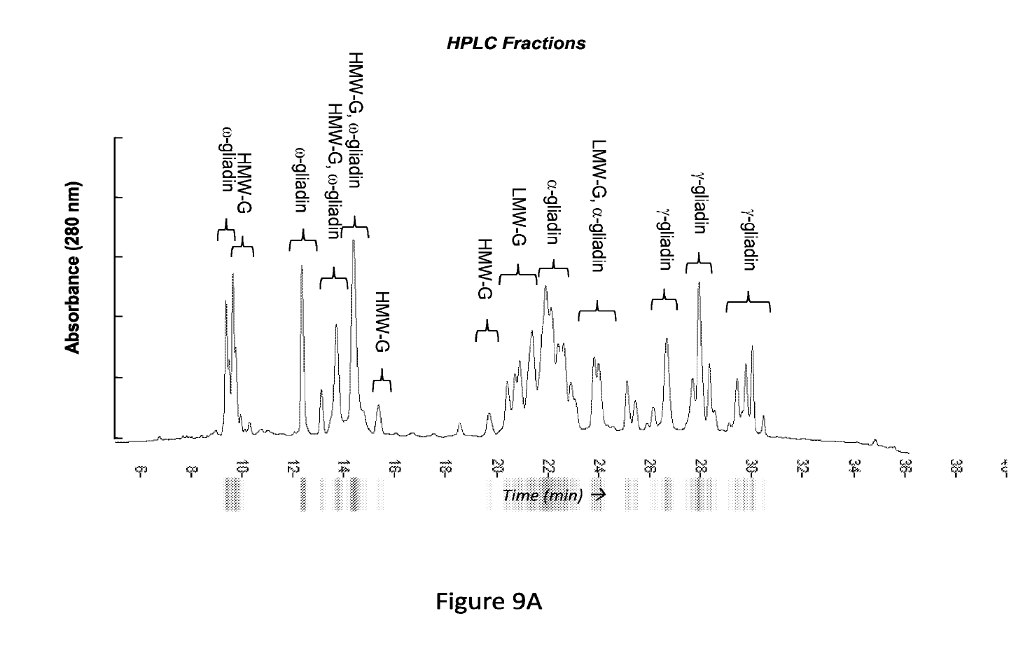

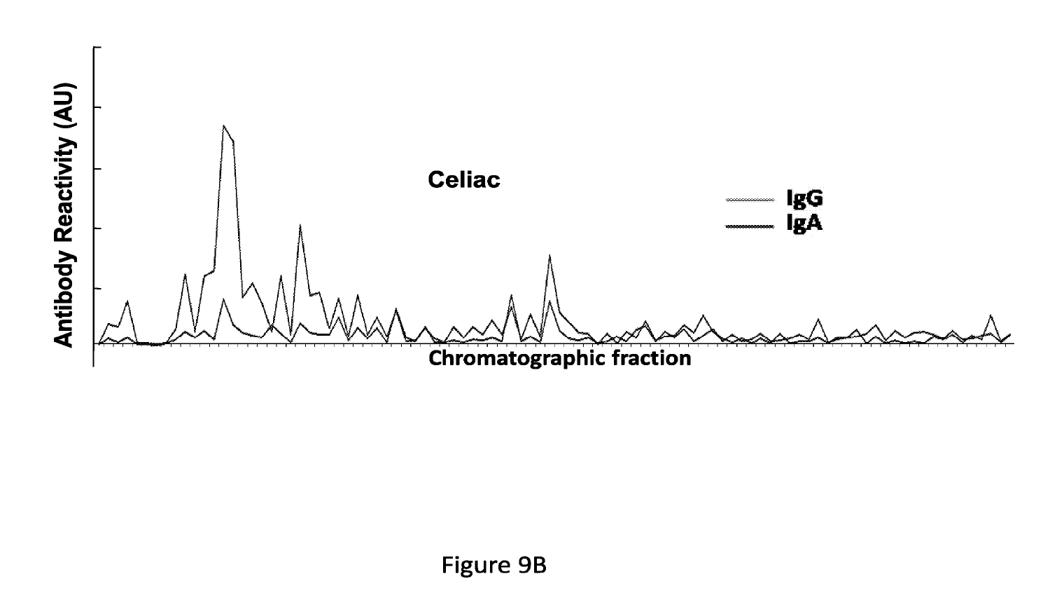

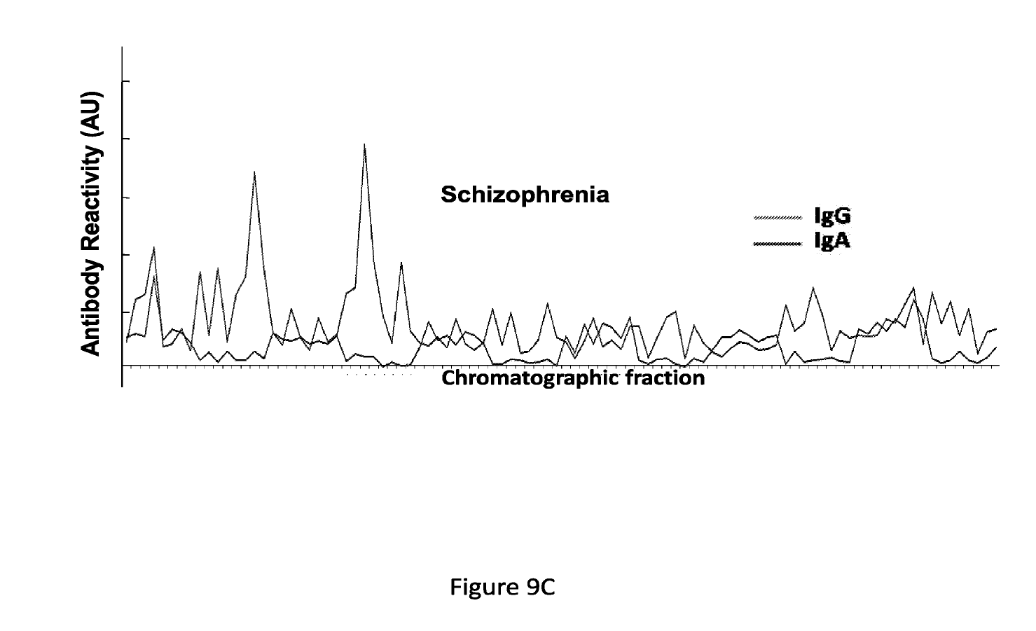

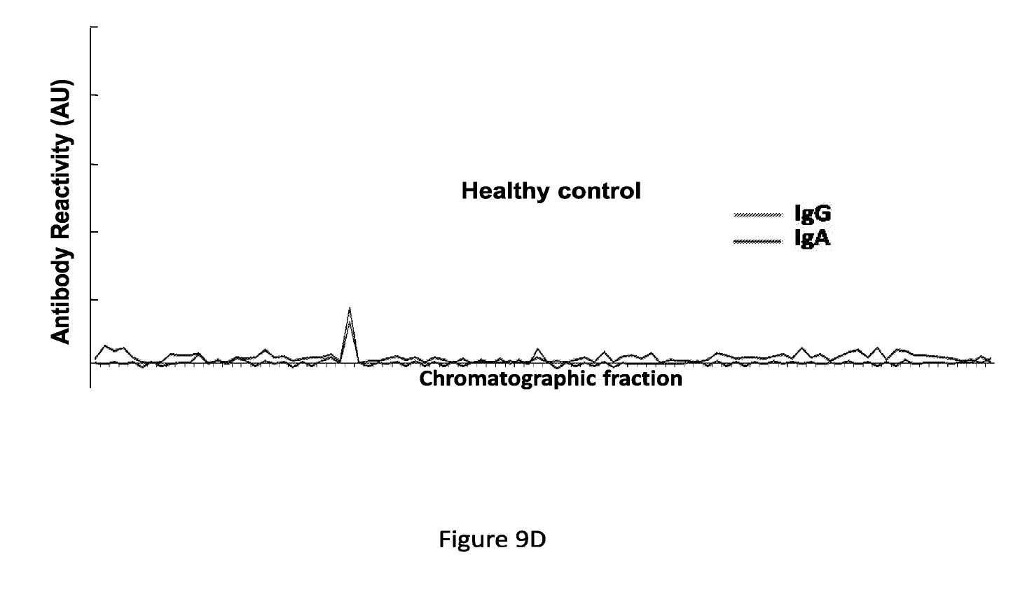

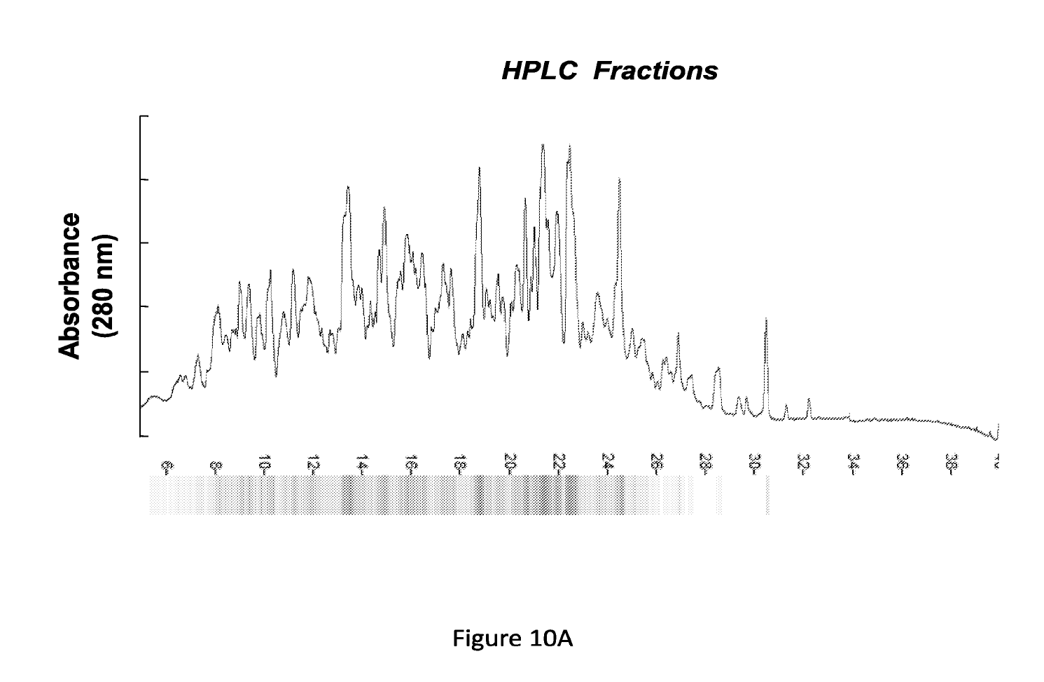

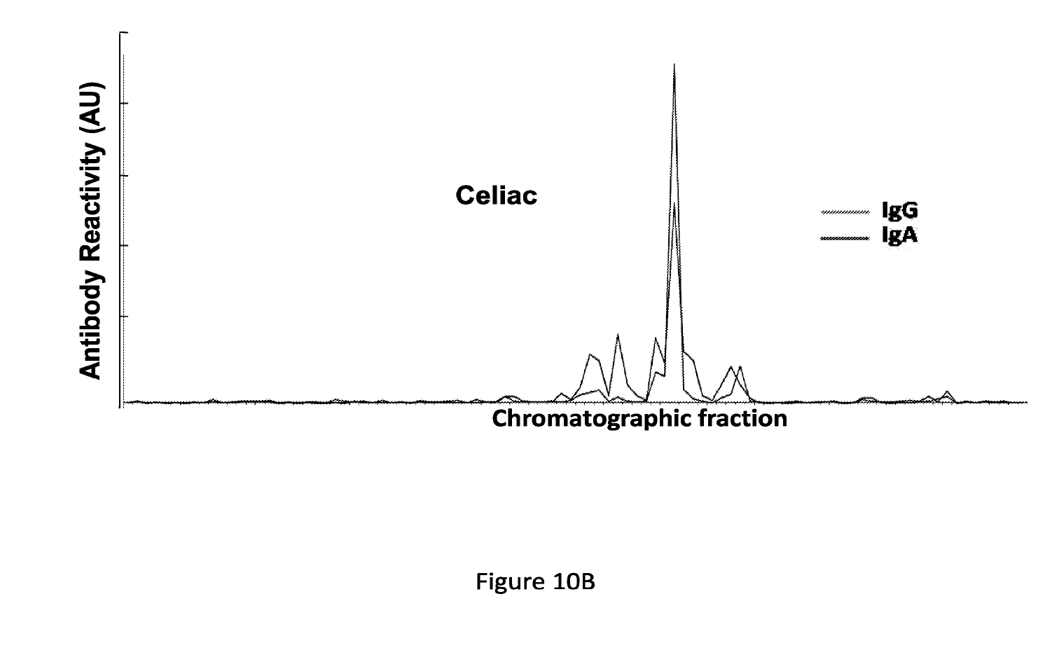

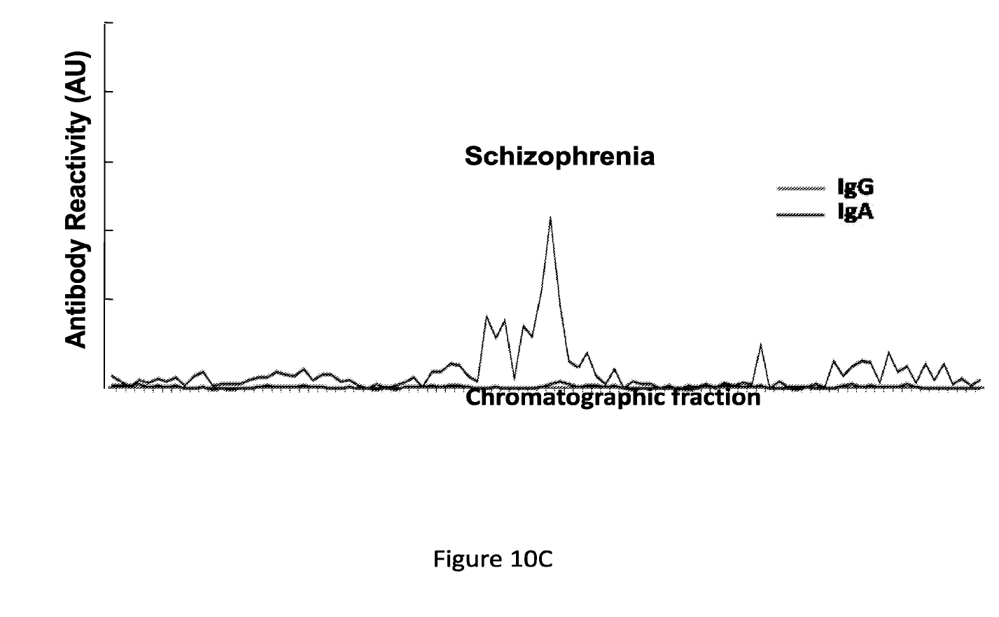

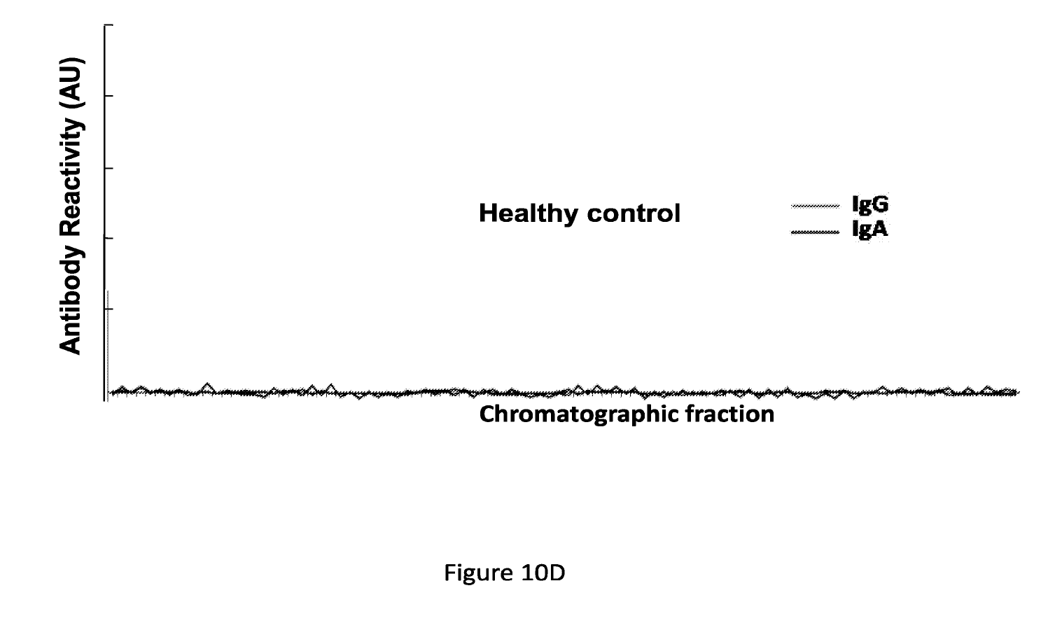

FIGS. 9 and 10 show analyzed fluorescence intensity data in relation to the chromatographic peaks from the gluten extract and the gluten digest preparations for the above patients. The data demonstrate how the array can be used to map the antibody profile in the immune response to wheat proteins in celiac disease and other gluten-related disorders.

### BIBLIOGRAPHY & REFERENCES

Adams et al., 2011, Gastrointestinal flora and gastrointestinal status in children with autism—comparisons to typical children and correlation with autism severity, BMC Gastroenterol. 11:22.
Alaedini et al., 2005, Narrative review: celiac disease: understanding a complex autoimmune disorder, Ann Intern Med. 142:289-298.
Alaedini et al., 2007, Immune cross-reactivity in celiac disease: anti-gliadin antibodies bind to neuronal synapsin I., J Immunol. 178:6590-6595.
Alaedini et al., 2008, Autoantibodies in celiac disease, Autoimmunity 41: 19-26.
Aldrimer et al., 2012, Reference intervals on the Abbot Architect for serum thyroid hormones, lipids and prolactin in healthy children in a population-based study, Scand J Clin Lab Invest. 72: 326-332.
Association, A. P., 1994, Diagnostic and Statistical Manual of Mental Disorders (DSM-IV). APA, Washington, D.C.
Barcia et al., 2008, Autism and coeliac disease, J Autism Dev Disord. 38:407-408.
Batista et al., 2012, Autism spectrum disorder and celiac disease: no evidence for a link, Arq Neuropsiquiatr. 70: 28-33.
Biesiekierski et al., 2011, Gluten causes gastrointestinal symptoms in subjects without celiac disease: a double-blind randomized placebo-controlled trial, Am J Gastroenterol. 106:508.
Briani et al., 2008, Celiac disease: from gluten to autoimmunity, Autoimmun Rev. 7:644-650.
Brown et al., 2011, Autoimmune and gastrointestinal dysfunctions: does a subset of children with autism reveal a broader connection, Expert Rev Gastroenterol Hepatol. 5: 465-477.
Cascella et al., 2009, Prevalence of Celiac Disease and Gluten Sensitivity in the United States Clinical Antipsychotic Trials of Intervention Effectiveness Study Population, Schizophr Bull. 37:94.
Cervio et al., 2007, Sera of patients with celiac disease and neurologic disorders evoke a mitochondrial-dependent apoptosis in vitro, Gastroenterology 133:195.
Chandra et al., 2011, Anti-Borrelia burgdorferi Antibody Profile in Post-Lyme Disease Syndrome, Clin Vaccine Immunol. 18:767.
Chandra et al., 2011, Epitope mapping of antibodies to V1sE protein of Borrelia burgdorferi in post-Lyme disease syndrome, Clin Immunol. 141:103.
Cunningham, 2010, Indirect immunofluorescent labeling of fixed cells. In Immunocytochemical Methods and Protocols, Methods in Molecular Biology, Vol. 588, C. Oliver, and M. C. Jamur, eds. Humana Press, p. 335.
D'Eufemia et al., 1996, Abnormal intestinal permeability in children with autism, Acta Paediatr. 85:1076-1079.
de Magistris et al., 2010, Alterations of the intestinal barrier in patients with autism spectrum disorders and in their first-degree relatives, J Pediatr Gastroenterol Nutr. 51:418-424.
Dickerson et al., 2010, Markers of gluten sensitivity and celiac disease in recent-onset psychosis and multi-episode schizophrenia, Biol Psychiatry. 68:100.
Dohan, 1969, Is celiac disease a clue to the pathogenesis of schizophrenia, Ment Hyg. 53:525-529.
DuPont et al., 2005, Sequential extraction and quantitative recovery of gliadins, glutenins, and other proteins from small samples of wheat flour, J Agric Food Chem. 53:1575.
Dupont et al., 2011, Deciphering the complexities of the wheat flour proteome using quantitative two-dimensional electrophoresis, three proteases and tandem mass spectrometry, Proteome Sci. 9:10.
Elder, 2008, The gluten-free, casein-free diet in autism: an overview with clinical implications, Nutr Clin Pract. 23:583-588.
First et al., 1998, Structured Clinical Interview for DSM-IV Axis I Disorders-Non-Patient Edition (SCID-UNP), In Biometrics Research New York State Psychiatric Institute, New York.
Frye et al., 2012, Cerebral folate receptor autoantibodies in autism spectrum disorder, Mol Psychiatry.
Goines et al., 2011, Autoantibodies to cerebellum in children with autism associate with behavior, Brain Behav Immun 25: 514-523.
Goodwin et al., 1969, In a dark mirror, Ment Hyg. 53: 550-563.
Greenlee et al., 2010, Purkinje cell death after uptake of anti-Yo antibodies in cerebellar slice cultures, J Neuropathol Exp Neurol. 69:997.
Hinson et al., 2007, Pathogenic potential of IgG binding to water channel extracellular domain in neuromyelitis optica, Neurology 69:2221.
Jabri et al., 2005, Innate and adaptive immunity: the yin and yang of celiac disease, Immunol Rev. 206: 219-231.
Jackson et al., 2011, Neurologic and Psychiatric Manifestations of Celiac Disease and Gluten Sensitivity, Psychiatr Q [Epub ahead of print].
Jin et al., 2010, A Study of Circulating Gliadin Antibodies in Schizophrenia Among a Chinese Population, Schizophr Bull [Epub ahead of print].
Kagnoff, 2007, Celiac disease: pathogenesis of a model immunogenetic disease, J Clin Invest. 117: 41-49.
Knapp et al., 2004, The global costs of schizophrenia, Schizophr Bull. 30:279.
Luckman et al., 2006, Effects of myasthenia gravis patient sera on human myoblast cultures, Acta Neurol Scand Suppl. 183:28.
Ludvigsson et al., 2013, The Oslo definitions for coeliac disease and related terms, Gut 62: 43-52.
Lundin et al., 2012, Non-celiac gluten sensitivity, Gastrointest Endosc Clin N Am. 22: 723-734.
McCarthy et al., 1979, Response of intestinal mucosa to gluten challenge in autistic subjects, Lancet 2: 877-878.
McEvoy, 2007, The costs of schizophrenia, J Clin Psychiatry 68 Suppl 14:4.
Mueser et al., 2004, Schizophrenia Lancet 363:2063.
NIH, National Institute of Mental Health. (http://www.nimh.nih.gov/). Updated 22 Sep. 2011.
Onore et al., 2012, The role of immune dysfunction in the pathophysiology of autism, Brain Behav Immun 26: 383-392.
Pavone et al., 1997, Autism and celiac disease: failure to validate the hypothesis that a link might exist, Biol Psychiatry 42: 72-75.
Qiao et al., 2012, The adaptive immune response in celiac disease, Semin Immunopathol. 34: 523-540.
Ruuskanen et al., 2011, Persistently positive gliadin antibodies without transglutaminase antibodies in the elderly: gluten intolerance beyond coeliac disease, Dig Liver Dis. 43:772.
Samaroo et al., 2010, Novel immune response to gluten in individuals with schizophrenia, Schizophr Res. 118:248-255.
Sapone et al., 2012, Spectrum of gluten-related disorders: consensus on new nomenclature and classification, BMC Med. 10:13.
Schwertz et al., 2004, Serologic assay based on gliadin-related nonapeptides as a highly sensitive and specific diagnostic aid in celiac disease, Clin Chem. 50: 2370-2375.
Shan et al., 2002, Structural basis for gluten intolerance in celiac sprue, Science 297:2275.
Sigal et al., 1997, A monoclonal antibody to Borrelia burgdorferi flagellin modifies neuroblastoma cell neuritogenesis in vitro: a possible role for autoimmunity in the neuropathy of Lyme disease, Infect Immun 65:1722.
Valicenti-McDermott et al., 2008, Gastrointestinal symptoms in children with an autism spectrum disorder and language regression, Pediatr Neurol. 39: 392-398.
van Eckert et al., 2006, Towards a new gliadin reference material—isolation and characterisation, J Cereal Sci. 43: 331-341.
Vojdani et al., 2004, Immune response to dietary proteins, gliadin and cerebellar peptides in children with autism, Nutr Neurosci. 7: 151-161.
Volta et al., 2011, Serological Tests in Gluten Sensitivity (Nonceliac Gluten Intolerance), J Clin Gastroenterol [Eupub ahead of print].
Wang et al., 2011, The prevalence of gastrointestinal problems in children across the United States with autism spectrum disorders from families with multiple affected members, J Dev Behav Pediatr. 32: 351-360.
Zhang et al., 2004, Anti-ganglioside antibody-mediated neuronal cytotoxicity and its protection by intravenous immunoglobulin: implications for immune neuropathies. Brain 127:1085.
Zhang et al., 2010, Mitochondrial DNA and anti-mitochondrial antibodies in serum of autistic children, J Neuroinflammation 7: 80.

What is claimed is:

1. A method of determining the molecular specificity of antibody response to gluten and non-gluten proteins of wheat in a group of patients, the method comprises the steps of:
(i) preparing a composition comprising gluten and non-gluten proteins from wheat;
(ii) generating a gluten microarray using the composition obtained in (i); and
(iii) generating profiles of antibody binding to target proteins or peptides on the array of (ii), wherein the antibodies are obtained from patients or control subjects, and the binding profile of antibodies from said patients as compared to those from control subjects will demonstrate the molecular specificity of antibody response to gluten and non-gluten proteins of wheat in said group of patients.

2. The method of claim 1, wherein the composition comprises extracts of gluten and non-gluten proteins from wheat.

3. The method of claim 1, wherein the composition comprises recombinant gluten and non-gluten proteins.

4. The method of claim 2, wherein the extracts are derived from intact gluten proteins or gluten digest.

5. The method of claim 2, wherein the extracts are derived from digesting the gluten or non-gluten proteins with one or more of pepsin, trypsin, and chymotrypsin.

6. The method of claim 2, wherein the extracts comprise a substance selected from the group consisting of intact proteins, proteins after enzymatic digestion, and peptides.

7. The method of claim 2, wherein the preparation of extracts comprises fractionation of proteins or peptides by high resolution chromatographic separation methods.

8. The method of claim 2, wherein the preparation of extracts comprises 2-D fractionation of proteins or peptides.

9. The method of claim 1, further comprises the step of identifying the target proteins or peptides by mass spectrometry-assisted peptide mass mapping.

10. The method of claim 9, further comprises the step of identifying antigenic determinants on the target proteins by epitope mapping.

11. The method of claim 1, wherein the patients display increased antibody response to wheat proteins.

12. The method of claim 1, wherein the patients are having a disease selected from the group consisting of celiac disease, wheat allergy, dermatitis herpetiformis, non-celiac gluten sensitivity, schizophrenia, bipolar disorder, autism, and sporadic ataxia.

13. The method of claim 1, wherein the target proteins or peptides are gluten proteins selected from the group consisting of gliadins and glutenins.

14. The method of claim 1, wherein the target proteins or peptides are non-gluten proteins selected from the group consisting of amylase inhibitors, trypsin inhibitors, serine protease inhibitors, purinins, globulins, and farinins.
